(19) United States
(12) Patent Application Publication
(10) Pub. No.: US 2013/0209485 A1
(43) Pub. Date: Aug. 15, 2013

(54) THERAPEUTIC AGENT OF NON-ALLERGIC AIRWAY INFLAMMATION AND/OR NON-ALLERGIC HYPERRESPONSIVE AIRWAY

(71) Applicant: RIKEN, (US)

(72) Inventors: Hiroshi WATARAI, Yokohama-shi (JP); Masaru TANIGUCHI, Yokohama-shi (JP)

(73) Assignee: RIKEN, Wako-shi (JP)

(21) Appl. No.: 13/624,238

(22) Filed: Sep. 21, 2012

Related U.S. Application Data
(60) Provisional application No. 61/553,544, filed on Oct. 31, 2011.

(51) Int. Cl. G01N 33/68; A61K 38/17; A61K 39/395

(52) U.S. Cl. CPC G01N 33/6869; A61K 39/3955; A61K 38/1793
USPC 424/158.1; 424/172.1; 514/1.1; 435/7.1; 435/7.92; 435/6.12; 435/6.11

(57) ABSTRACT

The invention provides a therapeutic agent for non-allergic airway inflammation and/or non-allergic airway hyperreactivity, containing a substance capable of inhibiting or eliminating the Th17 cell-like function of an IL-17RB positive NKT cells as an active ingredient; a prophylactic or therapeutic method for non-allergic airway inflammation and/or non-allergic airway hyperreactivity, using the substance, and a method of screening for a therapeutic agent for non-allergic airway inflammation and/or non-allergic airway hyperreactivity, containing a step of measuring a Th17 cell-like function of IL-17RB positive NKT cells.

### TECHNICAL FIELD OF THE INVENTION

The present invention relates to a novel method of screening a therapeutic agent for non-allergic airway inflammation and/or non-allergic airway hyperreactivity. More particularly, the present invention relates to a method of screening a therapeutic agent for non-allergic airway inflammation and/or non-allergic airway hyperreactivity including measuring the Th17 cell-like function of an IL-17RB positive NKT cells, and a method for the prophylaxis or treatment of non-allergic airway inflammation and/or non-allergic airway hyperreactivity, comprising administering an effective amount of a substance capable of inhibiting or eliminating the Th17 cell-like function of an IL-17RB positive NKT cells.

### BACKGROUND OF THE INVENTION

Respiratory diseases such as airway hyperreactivity, airway inflammation and asthma have an unclarified onset mechanism, and therefore, cure and remission thereof are difficult. Particularly, the onset mechanism thereof is complicated involving motility induction, aspirin, air pollution, virus infection and the like, and the pathology varies depending on the age. According to the classification of Swineford, they are largely divided into allergic type and non-allergic type. The allergic type is induced by allergen, Th2 cells and eosinophils play a central part in the formation of pathology, and NKT cells are involved in the onset thereof. On the other hand, the non-allergic type shows an infectious, motility inducive, drug-inducive and environmental pathogenic mechanism, wherein the pathogenic mechanism and the chronic inflammation mechanism thereof remain unknown.

Since the above-mentioned respiratory diseases show diversity in pathologies to be formed, symptomatic therapeutic control using substances having an immunity suppressive action such as corticosteroid, and bronchodilating agents such as β2 stimulants is the main treatment. To provide a more accurate treatment aiming at cure and remission, it is demanded to correctly understand the initial reaction of early stages of pathology formation, clarify the molecular mechanism thereof, and develop a medicament targeting same.

Natural killer T (NKT) cells are recognized and activated using glycosphingolipid, which is presented by antigen presenting molecule CD1d on an antigen presenting cells (APCs), as a ligand, and induces production and cytotoxic activity of both Th1/2 cytokines. It has been reported that NKT cells are involved in the remission of various diseases, but also involved in aggravation. Akbari O., et al. (Essential role of NKT cells producing IL-4 and IL-13 in the development of allergen-induced airway hyperreactivity. Nat Med. 2003 May; 9(5):582-588.) have reported for the first time that NKT cells produce IL-4 and IL-13, which are Th2 cytokines, and are involved in allergic airway hyperreactivity.

IL-17 is a glycoprotein of a homodimer consisting of a peptide having a molecular weight of 20 to 30 kD, and it is currently known that it consists of six family molecules having homology (IL-17, IL-17B, IL-17C, IL-17D, IL-25(IL-17E), IL-17F) in addition to IL-17 (also referred to as IL-17A).

IL-17 is produced mainly from an activated T cells, while an IL-17 receptor (hereinafter, also referred to as IL-17R) is constitutively expressed in various cells. It is known that, like a ligand, a receptor forms a family (IL-17RA, IL-17RB, IL-17RC, IL-17RD, IL-17RE). The deduced amino acid sequence of IL-17RB is shown by SEQ ID NO:67.

Pichavant M., et al. (Ozone exposure in a mice model induces airway hyperreactivity that requires the presence of natural killer T cells and IL-17. J. Exp. Med. 2008 205(2):385-393.) report that NKT cells and IL-17 are involved in the onset of airway hyperresponsiveness in an air pollution model using ozone. Kim E Y. et al. (Persistent activation of an innate immune response translates respiratory viral infection into chronic lung disease. Nat Med 2008 14(6):633-640.) report that NKT cells and macrophage play a key role in the onset of chronic airway inflammation due to virus infection.

Furthermore, the present inventors have reported that allergic airway inflammation by NKT cells can be induced by IL-17RB positive NKT cells, particularly, promoted Th2 cell-like function of the cells (WO2009/069355, Terashima A. et al. (A novel subset of mouse NKT cells bearing the IL-17 receptor B responds to IL-25 and contributes to airway hyperreactivity. J. Exp. Med. 2008 205(12):2727-2733.)).

However, there are many cases that cannot be explained solely by the involvement of Th2 cells and eosinophils, such as non-allergic asthma and the like.

### SUMMARY OF THE INVENTION

### Problems to be Solved by the Invention

An object of the present invention is to provide a screening method for obtaining a therapeutic agent for non-allergic airway inflammation and/or non-allergic airway hyperreactivity, a method for the prophylaxis or treatment of non-allergic airway inflammation and/or non-allergic airway hyperreactivity based on the novel action mechanism, and various means capable of developing them etc.

### Means of Solving the Problems

While the present inventors have reported that a cell population involved in the onset of the pathology of airway hyperreactivity and airway inflammation is NKT cells that express IL-17RB, they have found that the cell population, upon activation, produces not only Th2 cytokines such as IL-9, IL-13 and the like, but also Th17 cytokines (IL-17A, IL-22 etc.), thereby triggering the onset of not only eosinophilic inflammation but also neutrophilic inflammation and macrophage inflammation.

Furthermore, by investigating influence of the IL-17RB positive NKT cells on the Th17 cell-like function, it was found out that a therapeutic agent, a screening method, and a method for the prophylaxis or treatment, of non-allergic airway inflammation and/or non-allergic airway hyperreactivity can be carried out, resulting in completion of the present invention. That is, the present invention is as follows.

[1] A therapeutic agent for non-allergic airway inflammation and/or non-allergic airway hyperreactivity comprising, as an active ingredient, a substance capable of inhibiting or removing Th17 cell-like function of IL-17RB positive NKT cells.
[2] The agent according to the above-mentioned [1], wherein the substance capable of inhibiting or removing Th17 cell-like function of IL-17RB positive NKT cells is at least one kind selected from the group consisting of an antagonistic antibody and a low-molecular inhibitor to IL-17RB, an antagonistic antibody and a low-molecular inhibitor to IL-23R, an antibody to IL-25 and a soluble molecule of IL-17RB, and an antibody to IL-23 and a soluble molecule of IL-17RB.
[3] A method of screening for a therapeutic agent for non-allergic airway inflammation and/or non-allergic airway hyperreactivity, comprising the step of measuring the Th17 cell-like function of an IL-17RB positive NKT cells.
[4] A method of screening for a therapeutic agent for non-allergic airway inflammation and/or non-allergic airway hyperreactivity, comprising the steps of:

contacting an IL-17RB positive NKT cells with a test compound in the presence of a ligand (step 1),

measuring the Th17 cell-like function of the IL-17RB positive NKT cells contacted with the test compound and the Th17 cell-like function of an IL-17 RB positive NKT cells not being contacted with the test compound, and comparing the results (step 2), and

selecting a test compound significantly inhibiting the Th17 cell-like function as a therapeutic agent for non-allergic airway inflammation and/or non-allergic airway hyperreactivity (step 3).

[5] The method according to the above-mentioned [4], wherein the Th17 cell-like function is the ligand stimulation-dependent Th17 cytokine/chemokine producing ability.
[6] The method according to the above-mentioned [4] or [5], wherein the ligand is IL-23.
[7] The method according to the above-mentioned [4] or [5], wherein the ligand is IL-25.
[8] The method according to the above-mentioned [5], wherein the Th17 cytokine/chemokine is at least one kind selected from the group consisting of IL-17A, IL-17F, IL-21, and IL-22.
[9] A method of screening for a therapeutic agent for non-allergic airway inflammation and/or non-allergic airway hyperreactivity, comprising the steps of:

administering a test compound to a mammal other than human, having an IL-17RB positive NKT cells in the presence of a ligand of IL-17RB (step 1),

measuring the Th17 cell-like function of the mammal to which the test compound is administered, and the Th17 cell-like function of a mammal to which the test compound is not administered, and comparing the results (step 2), and

selecting a test compound significantly inhibiting the Th17 cell-like function as a therapeutic agent for non-allergic airway inflammation and/or non-allergic airway hyperreactivity (step 3).

[10] The method according to the above-mentioned [9], wherein the Th17 cell-like function is the ligand stimulation-dependent Th17 cytokine/chemokine producing ability.
[11] The method according to the above-mentioned [9] or [10], wherein the ligand is IL-23.
[12] The method according to the above-mentioned [9] or [10], wherein the ligand is IL-25.
[13] The method according to the above-mentioned [10], wherein the Th17 cytokine/chemokine is at least one kind selected from the group consisting of IL-17A, IL-17F, IL-21, and IL-22.
[14] A method of preventing or treating non-allergic airway inflammation and/or non-allergic airway hyperreactivity, comprising administering an effective amount of a substance capable of inhibiting or removing Th17 cell-like function of IL-17RB positive NKT cells to a mammal.
[15] The method according to the above-mentioned [14], wherein the substance capable of inhibiting or removing Th17 cell-like function of IL-17RB positive NKT cells is at least one kind selected from the group consisting of an antagonistic antibody and a low-molecular inhibitor to IL-17RB, an antagonistic antibody and a low-molecular inhibitor to IL-23R, an antibody to IL-25 and a soluble molecule of IL-17RB, and an antibody to IL-23 and a soluble molecule of IL-17RB.

### Effect of the Invention

Since an activated IL-17RB positive NKT cells have the Th17 cell-like function from results of analysis of the function thereof and produce a large amount of a Th17 cytokine such as IL-17A, IL-22 etc. by stimulation of a ligand (IL-23 or IL-25), it is thought that it has a central role in exacerbation of non-allergic airway hyperreactivity or non-allergic airway inflammation. Previously, regarding a respiratory disease such as asthma etc., symptomatic therapy using a steroid has been performed, but the case of steroid unresponsiveness is also known, and correlation with the case in which an NKT cells are involved in exacerbation has been pointed out. From these results, it is expected that the respiratory disease can be treated by inhibiting or eliminating the function of the IL-17RB positive NKT cells.

### BRIEF DESCRIPTION OF THE DRAWINGS

FIG. 1 shows the construction of a targeting vector of IL-17RB.

FIG. 2A shows the number of NKT cells in IL-17RB deficient mice (C57BL/6 background) and IL-15 mutant mice (spleen, liver).

FIG. 2B shows the number of IL-17RB positive NKT cells in IL-17RB deficient mice (C57BL/6 background) and IL-15 mutant mice (spleen, liver).

FIG. 2C shows the results of cytokine production in NKT cells derived from the spleen of IL-17RB deficient mice and IL-15 mutant mice.

FIG. 3A shows the number of NKT cells in IL-17RB deficient mice (C57BL/6 background) and IL-15 mutant mice (thymus).

FIG. 3B shows distribution of NKT cells in 3 stages of thymic differentiation: CD44 negative NK1.1 negative (stage 1), CD44 positive NK1.1 negative (stage 2), CD44 positive NK1.1 positive (stage 3) (IL-17RB deficient mice-derived NKT cells, IL-15 mutant mice-derived NKT cells).

FIG. 3C shows the results of expression of IL-17RB and CD122 (IL-15 receptor β chain) in the thymic differentiation stages of NKT cells.

FIG. 3D shows the results of expression of IL-17RB and CD4 in IL-15 mutant mice-derived NKT cells.

FIG. 3E shows the results of expression of CD4 and IL-17RB in the thymic differentiation stages of wild-type mice and IL-15 mutant mice-derived NKT cells.

FIG. 3F is a graph showing the summary of expression of CD4 and IL-17RB in the thymic differentiation stages of wild-type mice, IL-17RB deficient mice and IL-15 mutant mice-derived NKT cells.

FIG. 4A shows the results of gene expression profile of wild-type mice (C57BL/6) thymic NKT cells by DNA microarray.

FIG. 4B shows the results of gene expression profile of wild-type mice and IL-15 mutant mice-derived NKT cells by DNA microarray.

FIG. 5 shows the results of cytokine production in NKT cells derived from the thymus of IL-17RB deficient mice and IL-15 mutant mice.

FIG. 6A shows the results of expression of CD4 gene (Cd4), IL-17RB gene (Il17rb), and IL-2RB(=Cd122) gene (Il2rb), examined by quantitative PCR, in each subtype of NKT cells obtained by dividing into 4 fractions using the expression of CD4 and IL-17RB on the cell surface as an index.

FIG. 6B shows the results of expression of Th1/Th2/Th17-related gene in each subtype of NKT cells obtained by dividing into 4 fractions using the expression of CD4 and IL-17RB on the cell surface as an index.

Th1-related gene: IFN-γ gene (Ifng), T-bet gene (Tbx21), Stat4 gene (Stat4)

Th2-related gene: IL-4 gene (Il4), Gata3 gene (Gata3)

Th17-related gene: IL-17A gene (Il17a), IL-22 gene (Il22), RORγt gene (Rorc)

FIG. 6C is a graph showing the results of cytokine production by coculture of each subtype of NKT cells in a test tube in the presence of bone marrow-derived dendritic cell and α-GalCer.

FIG. 6D shows the results of expression of IL-12 receptor (IL-12RB1, IL-12RB2), IL-23 receptor (IL-23R, IL-12RB1), IL-25 receptor (IL-17RA, IL-17RB (FIG. 6A)) in each subtype of NKT cells as examined by quantitative PCR.

FIG. 6E shows the results of cytokine reactivity of each subtype of NKT cells (in the presence of bone marrow-derived dendritic cell and IL-12).

FIG. 6F shows the results of cytokine reactivity of each subtype of NKT cells (in the presence of bone marrow-derived dendritic cell and IL-25).

FIG. 6G shows the results of cytokine reactivity of each subtype of NKT cells (in the presence of bone marrow-derived dendritic cell and IL-23).

FIG. 6H shows the measurement results of expression of chemokine receptors in each subtype of NKT cells.

FIG. 7A shows the measurement results of the content of each subtype of NKT cells in thymus NKT cells depending on the mice lineage.

FIG. 7B shows the results of gene expression profile of wild-type mice (Balb/c) thymic NKT cells by DNA microarray.

FIG. 7C shows the results of gene expression profile of wild-type mice (C57BL/6 and Balb/c) thymic NKT cells by DNA microarray.

FIG. 8A shows the results of peripheral localization of each subtype of NKT cells. Examined were spleen, liver, bone marrow, lung, inguinal lymph node, and mesenteric lymph node of wild-type mice and IL-17RB deficient mice (C57BL/6).

FIG. 8B shows the results of peripheral localization of each subtype of NKT cells. Examined were spleen, liver, bone marrow, lung, inguinal lymph node, and mesenteric lymph node of wild-type mice and IL-17RB deficient mice (C57BL/6).

FIG. 8C shows the analysis results of the contents of 4 NKT cell subtypes, divided by the expression of CD4 and IL-17RB, in each organ (C57BL/6 and Balb/c).

FIG. 9A shows the results of peripheral localization of each subtype of NKT cells. Examined were spleen, liver, bone marrow, lung, inguinal lymph node, and mesenteric lymph node of wild-type mice and IL-17RB deficient mice (Balb/c).

FIG. 9B shows the analysis results of the contents of 4 NKT cell subtypes, divided by the expression of CD4 and IL-17RB, in each organ (Balb/c).

FIG. 10A shows the results of gene expression profile of NKT cells derived from the thymus and spleen of wild-type mice (C57BL/6) by DNA microarray.

FIG. 10B shows the results, regarding thymic NKT cells, of confirmed expression of CD4 and IL-17RB in NKT cells present in the spleen of NKT cell deficient mice (Jα18 deficient mice) after transfer of each subtype of NKT cells obtained by dividing into 4 fractions using the expression of CD4 and IL-17RB on the cell surface as an index.

FIG. 11A shows the number of NKT cells in each organ of IL-15 mutant mice.

FIG. 11B shows the number of each subtype of NKT cells in each organ of IL-15 mutant mice.

FIG. 12A shows the results of RORγt induction in each subtype of NKT cells obtained by dividing into 4 fractions using the expression of CD4 and IL-17RB on the cell surface as an index.

FIG. 12B shows the results of induction of transcription factor E4 bp4 in each subtype of NKT cells obtained by dividing into 4 fractions using the expression of CD4 and IL-17RB on the cell surface as an index.

FIG. 12C shows the results of cytokine production due to the stimulation, with IL-25, of CD4 positive IL-17RB positive NKT cells derived from the thymus and spleen of wild-type mice (C57BL/6) and E4 bp4 deficient mice.

FIG. 13 is a schematic showing of the structure of RS virus membrane type G protein.

FIG. 14A is a schematic showing of the steps of preparation of RS virus transnasally infected model.

FIG. 14B shows the measurement results of methacholine inducive airway pressure in each animal model.

FIG. 14C shows the analysis results of infiltrated cells in bronchoalveolar washing of each animal model infected with RS virus.

FIG. 14D shows the observation results of cell infiltration in bronchoalveolar washing and excessive production of mucin of each animal model infected with RS virus.

### DESCRIPTION OF EMBODIMENTS

Unless otherwise indicated in sentences, all technical terms and scientific terms used herein have the same meanings as those that are generally understood by a person skilled in the technical field to which the present invention belongs. Arbitrary methods and materials which are same as or equivalent to those described in the present specification can be used in implementation or tests of the present invention, and preferable methods and materials are described below. All publications and patents referred in the present specification are incorporated herein by reference, for the purpose of describing and disclosing the constructs and methodologies, described in the publications which are usable with reference to the described inventions.

It is the finding obtained in the present invention that IL-17RB positive NKT cells are activated by the stimulation with a ligand to induce a Th17 cell-like function.

NKT cells are assembly of functionally different cell populations. When classified using the surface antigen of IL-17RB and CD4 as a marker, they are classified into the following 4: (i) CD4 negative IL-17RB positive (CD4⁻IL-17RB⁺), (ii) CD4 positive IL-17RB positive (CD4⁺IL-17RB⁺), (iii) CD4 negative IL-17RB negative (CD4⁻IL-17RB⁻), (iv) CD4 positive IL-17RB negative (CD41L-17RB⁻).

The present invention provides a method of screening for a therapeutic agent for non-allergic airway inflammation and/or non-allergic airway hyperreactivity, comprising measuring the Th17 cell-like function of IL-17RB positive NKT cells, particularly an IL-17RB positive NKT cells activated by stimulation with a ligand. The non-allergic airway inflammation and non-allergic airway hyperreactivity are inflammatory diseases characterized by contraction of trachea in response to stimulation of causes other than allergy (e.g., hyperkinesis, drugs such as aspirin and the like, virus infection, air pollution etc.), and sometimes accompanies asthmatic attack. Asthmatic attack may occur by inhaling cold air or running suddenly, since the trachea excessively reacts to even a small stimulation.

In the present invention, “Th17 cell-like function” means a function equivalent to that of Th17 cells, which is a subtype of T cells that produce cytokines such as IL-17A, IL-17F, IL-21, IL-22 and the like. Specifically, it means an ability of activated IL-17RB positive NKT cells to produce IL-17A, IL-17F, IL-21, IL-22 and the like, and various actions that can be exhibited by various cytokines and chemokines produced.

Furthermore, IL-17RB positive NKT cells activated by stimulation with ligand produce cytokines to be aggravation factors such as IL-17 and the like, and show a function of chemotaxis to the local site of Th2 cells and neutrophils.

IL-17A (interleukin-17A) is an inflammation inducing cytokine, which promotes antigen stimulation by T cells, and stimulation to macrophages, fibroblasts, endothelial cells, and outer skin cells, to produce inflammation mediators such as IL-1, IL-6, TNF-α, NOS-2, metalloprotease and chemokine. IL-17 is known to transmit signals via IL-17 receptor.

IL-17F (interleukin-17F) is known to have a biological activity similar to that of IL-17A, act on various cells such as fibroblasts, epithelial cells, vascular endothelial cells and the like to induce various inflammatory mediators such as antibacterial peptides, cytokines, chemokines, and matrix metalloproteases.

IL-21 (interleukin-21) is strongly produced by follicular helper T cells and Th17 cells, whereas IL-21 is under autocrine regulation and acts to sustain differentiation into Th17 cells.

IL-22 (interleukin-22) is also known as a T cell-derived inducible factor related to IL-10. IL-22 is mainly produced by activated T cells and NK cells.

Therefore, the measurement of the Th17 cell-like function of IL-17RB positive NKT cells includes measurement of the ability to produce ligand stimulation-dependent Th17 cytokine/chemokine (IL-17A, IL-17F, IL-21, IL-22 etc.), measurement of the presence or absence of ligand stimulation-dependent increase in airway resistance, measurement of the number of lymphocytes contained in a bronchoalveolar washing solution accompanied therewith and the like. Detailed measurement methods and measurement procedures are described later in the Examples.

IL-17RB positive NKT cells to be used for this screening method can be obtained by detecting and separating NKT cells expressing IL-17RB from a cell population containing NKT cells by using an antibody to IL-17RB.

The antibody against IL-17RB is not particularly limited as long as it specifically recognizes IL-17RB. Here, “specifically recognizes” means that the antibody immunologically cross-reacts with an epitope possessed solely by IL-17RB molecule, and does not cross-reacts with proteins of other family. The antibody may be any of peptide antibody, polyclonal antibody and monoclonal antibody.

Specifically, the antibody described in WO2009/069355 is used.

Detection of IL-17RB is not limited to detection with FACS. For example, it is predicted that IL-17RB can be detected by acting the present antibody as a 1st antibody in Western blotting, and expression at a protein level can be confirmed. Alternatively, the present antibody is bound to a solid phase (polystyrene beads, microtiter well surface, latex beads etc.), an immunological reaction is performed in a heterogeneous system or a homogeneous system, and IL-17RB can be detected and quantitated (using a method such as a fluorescent antibody method, ELISA, radioimmunoassay etc.). In this case, the immunological reaction may be a competition reaction or a non-competition reaction. Alternatively, a reaction by a sandwich method using two or more antibodies (monoclone or polyclone) may be used. For the detection and the quantitation, any immunological procedures known in the art can be used.

NKT cells are one kind of lymphocytes which are small in its existence ratio, but have a controlling role in immune system. NKT cells have two antigen receptors of a T cell receptor (TCR) and an NK receptor. An origin of the NKT cells is not particularly limited, but the NKT cells can be recovered from umbilical blood, peripheral blood, lung, bone marrow, spleen, lymph node, thymus gland etc. of a mammal such as a primate including human, a rodent, rabbit, cat, dog, horse, cow, sheep, goat, and pig. A term “primate” used herein means an arbitrary animal belonging to a group of a mammal, which is not limited to, but includes monkey, ape and human. Specifically, a suspension of a single cell recovered from a homogenate of peripheral blood, lung, spleen or thymus gland is selected and recovered by FACS analysis using an antigenic glycolipid such as α-galactosylceramide (α-GalCer or α-GC) presented on a CD1d molecule, which can be recognized by TCR highly restricted in NKT cells, and then only the NKT cells expressing IL-17RB are selected and recovered using the aforementioned antibody to IL-17RB. Alternatively, since IL-17RB is specifically expressed in a part of the NKT cells in the steady state, an IL-17RB positive NKT cells can be also selected and recovered from a suspension of a single cell recovered from a homogenate of peripheral blood, lung, spleen, or thymus gland, preferably direct from a leukocyte derived from spleen using an antibody to IL-17RB.

Moreover, both subsets of CD4 negative IL-17RB positive NKT cells and IL-17RB positive NKT cells of CD4 positive IL-17RB positive NKT cells can be selected and recovered by repeating the analysis using antibody to CD4 (e.g., FACS analysis). Here, the antibody to CD4 can be produced by a well known method, or by appropriately modifying an antibody based thereon, or commercially available antibody can also be used.

One aspect (aspect 1) of a method of screening a therapeutic agent for non-allergic airway inflammation and/or non-allergic airway hyperreactivity of the present invention will be shown below.

### Aspect 1

(Step 1) A step of contacting an IL-17RB positive NKT cells with a test compound in the presence of a ligand of IL-17RB.

Examples of the IL-17RB positive NKT cells used in the present step include cells which were detected and separated with the antibody to IL-17RB. Preferred is a CD4 negative cells separated using an antibody to CD4. The NKT cells are preferably activated by APC, or are used in the condition under which the cell is activated. Specifically, the present step is performed in the presence of APC. A ligand used in the present step is not particularly limited as far as it is a substance which acts on the IL-17RB positive NKT cells to induce Th17 cell-like function, that is, produces Th17 cytokines or chemokines, but it is preferable to use IL-23 known to induce differentiation of Th17 cells. IL-25 as a ligand can be also used preferably. A concentration of the ligand used is arbitrarily set in such a range that the ligand does not disadvantageously act on proliferation of the IL-17RB positive NKT cells, preferably the ligand can promote proliferation to produce a Th17 cytokines or chemokines, and is usually 0.1 to 10 ng/ml, preferably around 1 ng/ml in a culturing solution or a buffer which becomes a medium of a reaction system. Herein, the ligand may be added to the reaction system before contact of a test compound with the IL-17RB positive NKT cells, or may be added to the reaction system after contact, or may be added to a reaction system simultaneously with the contact, as far as the ligand is present in the reaction system in the state where it acts on the IL-17RB positive NKT cells to produce Th17 cytokines or chemokines.

In the present specification, the “test compound” is a compound which was selected or synthesized for the purpose of investigating whether it can act on the IL-17RB positive NKT cells to inhibit or eliminate the function thereof or not, and also includes the known compound which has already been reported to have other action, in addition to a novel compound. Alternatively, the test compound may be used as a composition. Examples include a nucleic acid (e.g. nucleoside, oligonucleotide, polynucleotide), a carbohydrate (e.g. monosaccharide, disaccharide, oligosaccharide, polysaccharide), a lipid (e.g. saturated or unsaturated straight, branched and/or ring-containing fatty acid), an amino acid, a protein (e.g. oligopeptide, polypeptide), an organic low-molecular compound, a compound library made using the combinatorial chemistry technique, a random peptide library made by solid phage synthesis or a phage display method, a natural component (e.g. compounds derived from microorganism, animal and plant, marine organism etc.), food, drinkable water etc. A compound which was recognized to have the action of inhibiting or eliminating the function of the IL-17RB positive NKT cells by the screening method of the present invention is expected to apply to non-allergic airway inflammation or non-allergic airway hyperreactivity as described above.

A method of contacting the IL-17RB positive NKT cells with the test compound is not particularly limited, as far as whether the test compound influences the ability to produce Th17 cytokine/chemokine possessed by an activated IL-17RB positive NKT cells or not can be determined, but the contact is simply performed by adding a predetermined amount in the presence of a ligand, in a reaction system such as a cell suspension containing an IL-17RB positive NKT cells and its activating substance (e.g. ligand and antigen presenting cells). An additive amount of the test compound is arbitrarily set depending on the situation of the cells, and usually it is preferable to set a dilution series. A period of time required for the contact can be arbitrarily set in such a range that the desired effect is obtained, and is usually 24 to 120 hours, preferably around 48 to 72 hours.

(Step 2) A step of measuring the Th17 cell-like function of the IL-17RB positive NKT cells contacted with the test compound and the Th17 cell-like function of an IL-17RB positive NKT cells not being contacted with the test compound, and comparing the results.

It is preferable that the IL-17RB positive NKT cells have been activated by an antigen presenting cell (APC) etc. By stimulating such the IL-17RB positive NKT cells with a ligand (e.g. IL-23, IL-25), the Th17 cell-like function is induced. Examples of the Th17 cell-like function specifically include the ability to produce Th17 cytokines/chemokines. Examples of the Th17 cytokines/chemokines include IL-17A, IL-17F, IL-21, IL-22 etc. as described above. The ability to produce these cytokines/chemokines can be usually measured by the method which is performed in the art, for example, Western blotting and an ELISA method using an antibody to each cytokines/chemokines, Northern blotting using a probe, and quantitative PCR using primers. Various antibodies, probes and primers can be arbitrarily prepared based on an amino acid sequence or a gene sequence of each cytokines/chemokines, or the known information such as a purification method etc., or are commercially available.

(Step 3) A step of selecting a test compound significantly inhibiting the Th17 cell-like function as a therapeutic agent for non-allergic airway inflammation and/or non-allergic airway hyperreactivity.

As in the Step 2, examples of the Th17 cell-like function include the ability to produce the Th17 cytokines/chemokines. It is thought that the Th17 cytokines/chemokines produced by IL-17RB positive NKT cells have a central role in exacerbation of airway hyperreactivity or asthma via, no allergic reaction, and therefore a test compound significantly inhibited the Th17 cell-like function, particularly inhibited production of the Th17 cytokines/chemokines, and selected based on the information obtained in the Step 2 can be a candidate of a therapeutic agent for non-allergic airway inflammation and/or non-allergic airway hyperreactivity. Significance of the effect can be usually determined by performing a significance test based on statistical treatment carried out in the art.

Further, another aspect (aspect 2) of the screening method provided by the present invention is indicated below.

### Aspect 2

(step 1) a step of administration of test compound in an environment of developing non-allergic airway inflammation and/or non-allergic airway hyperreactivity in mammal other than human, which has IL-17RB positive NKT cells (step 1).

A mammal other than a human having the IL-17RB positive NKT cells used in the present step is not particularly limited as far as it is a mammal other than a human, for which the action such as ligand stimulation-dependent production of Th17 cytokines/chemokines and increase in an airway resistance etc. is recognized, and the examples thereof include a mammal such as a primate other than a human, a rodent, rabbit, cat, dog, horse, cow, sheep, goat, and pig. The environment that develops non-allergic airway inflammation and/or non-allergic airway hyperreactivity includes, but is not limited to, an environment with induced production of Th17 cytokines/chemokines by hyperkinesis, drugs such as aspirin and the like, virus infection, air pollution etc. and/or ligand stimulation. Examples of the ligand and the test compound include the same ligand and test compound as those used in the aspect 1 of the screening method of the present invention.

One embodiment of the environment where non-allergic airway inflammation and/or non-allergic airway hyperreactivity are/is developed includes an environment where infection with RS virus, which plays a central role in the onset of virus asthma, occurred during childhood. Such environment can be constructed by allowing transnasal infection with RS virus several times, and further administering soluble G protein (Gs) considered to be responsible for RS virus pathogenesis and an antigen glycolipid α-GalCer.

To be specific, said such environment can be constructed by administration of 1-100 μg of Gs protein and 0.2-2 μg of α-GalCer. The order of administration of Gs protein and α-GalCer is not particularly limited as long as such environment can be constructed. Preferably, they are simultaneously administered.

The order of the step to administer the test compound to a mammal other than human and the step to construct an environment where non-allergic airway inflammation and/or non-allergic airway hyperreactivity are/is developed is not particularly limited, and the administration of the test compound may be performed before or after construction of the environment where non-allergic airway inflammation and/or non-allergic airway hyperreactivity are/is developed.

The dose of the ligand is not particularly limited as far as it is such an amount as to induce the Th17 cell-like function ligand stimulation-dependently, for an IL-17RB positive NKT cells (preferably, in the activated state) present in a body of a mammal other than a human, that is, as to produce Th17 cytokines/chemokines (IL-17A, IL-17F, IL-21, IL-22 etc.), and/or as to lead to increase in an airway resistance by an airway constricting substance (methacholine etc.) stimulation, and is usually 1 to 100 mg, preferably around 1 to 10 mg per 1 kg weight. An order of administering the test compound and the ligand to a mammal other than a human is not particularly limited as far as the Th17 cell-like function of IL-17RB positive NKT cells is induced, and influence of the test compound on the function can be measured, and administration of the test compound may be before administration of the ligand, or after administration of the ligand.

In addition, timing for administration can be arbitrary studied, depending on an action point which is desired as a therapeutic agent for non-allergic airway inflammation and/or non-allergic airway hyperreactivity, particularly a therapeutic agent for non-allergic asthma.

(Step 2) A step of measuring the Th17 cell-like function of the mammal to which the test compound is administered and a mammal to which a test compound is not administered, and comparing the results.

In the present step, examples of the “Th17 cell-like function” include the same functions as those described in the aspect 1 of the screening method of the present invention. Specifically, the examples include measurement of the ability to produce Th17 cytokines/chemokines (IL-17A, IL-17F, IL-21, IL-22 etc.), measurement of the presence or the absence of increase in an airway resistance, measurement of the number of lymphocyte contained in a bronchoalveolar washing solution accompanied with this, etc. Measurement of the ability to produce Th17 cytokines/chemokines is carried out by the same method as that described in the aspect 1 of the screening method of the present invention. The presence or the absence of increase in an airway resistance can be measured using an asthma model. The asthma model used in the present invention is a non-allergic type model, and its onset mechanism such as infectiousness, motility induction, drug-induction, environment and the like are not particularly questioned. For example, it may be an RS virus infected animal shown in the below-mentioned Example 6. Since the object is to measure the Th17 cell-like function of the IL-17RB positive NKT cells using this model, ligand is preferably used for inducing the Th17 cell-like function of the IL-17RB positive NKT cells. However, it does not apply when a spontaneous onset model is used. A spontaneous onset model is preferably used since it is more convenient. By the inducement, neutrophilic infiltration is recognized at a periphery of bronchus or at a periphery of a blood vessel. When airway constriction is caused by methacholine etc., there is a very strong reaction, and an airway resistance is increased. The Th17 cell-like function of the IL-17RB positive NKT cells is measured based on an extent of this increase in an airway resistance. By measuring whether the test compound inhibits this increase in the airway resistance or not, usefulness of the test compound as a therapeutic agent for non-allergic airway inflammation and/or non-allergic airway hyperreactivity can be confirmed. The number of macrophages and lymphocytes contained in the bronchoalveolar washing can be measured by a method used in the field of general clinical tests. For example, infiltration of the cell such as neutrophils and the like can be confirmed by PAS staining. By measuring whether the test compound inhibits, for example, increase in the number of the neutrophils in the bronchoalveolar washing solution or not, usefulness of the test compound as a therapeutic agent for non-allergic airway inflammation and/or non-allergic airway hyperreactivity can be confirmed.

(Step 3) A step of selecting a test compound significantly inhibiting the Th17 cell-like function as a therapeutic agent m for non-allergic airway inflammation and/or non-allergic airway hyperreactivity.

The present step can be performed as in the step 3 of the aspect 1 of the screening method of the present invention.

In the screening method of the present invention, preferably, a positive control compound can be used. The positive control compound is a compound known to inhibit the Th17 cell-like function of the IL-17RB positive NKT cells, that is, the ability to produce Th17 cytokines or chemokines by stimulation with the ligand, in advance. Specifically, examples include an antagonistic antibody and a low-molecular inhibitor to IL-17RB, an antibody to IL-25, and a soluble molecule of IL-17RB. Also, as a compound inhibiting the Th17 cell-like function of the CD4 negative cells among the IL-17RB positive NKT cells, an antagonistic antibody and a low-molecular inhibitor to IL-23R, an antibody to IL-23, and a soluble molecule of IL-23R can be mentioned.

The antagonistic antibody to IL-17RB or IL-23R inhibits or eliminates the function of the IL-17RB positive NKT cells by antagonistically acting on IL-17RB or IL-23R which is a target antigen, or inhibiting binding of IL-17RB or IL-23R and a ligand thereof, and the antibody to IL-25 or IL-23 inhibits or eliminates the function by inhibiting binding of IL-17RB or IL-23R and a ligand thereof. Examples of the low-molecular inhibitor to IL-17RB or IL-23R include a low-molecular substance capable of regulating interaction between IL-17RB or IL-23R and a ligand thereof to inhibit or eliminate the Th17 cell-like function of the IL-17RB positive NKT cells, and a low-molecular substance capable of regulating an intracellular signal pathway involving IL-17RB or IL-23R and a ligand thereof to inhibit or eliminate the Th17 cell-like function of the IL-17R8 positive NKT cells. Further, a soluble molecule (i.e. a molecule corresponding to an extracellular region) of IL-17RB or IL-23R inhibits or eliminates the function of the IL-17RB positive NKT cells, by competitively binding to the ligand. A used concentration of the positive control compound is not particularly limited, as far as it is such a concentration that the action of inhibiting or eliminating the function of the IL-17RB positive NKT cells is confirmed, but the concentration is different depending on a kind of a compound used and, for example, in the case of using the antagonistic antibody to IL-17RB or IL-23R, the concentration is usually 1 to 100 mg, preferably around 1 to 10 mg per 1 kg weight.

As the antagonistic antibody and the low-molecular inhibitor to IL-17RB or IL-23R, the antibody to IL-25 or IL-23, and the soluble molecule of IL-17RB or IL-23R, the same substances as those that can inhibit or eliminate the Th17 cell-like function of the IL-17RB positive NKT cells, contained in a therapeutic agent for non-allergic airway inflammation and/or non-allergic airway hyperreactivity described later are used, respectively.

The present invention also provides a therapeutic agent for non-allergic airway inflammation and/or non-allergic airway hyperreactivity comprising, as an active ingredient, a substance capable of inhibiting or eliminating Th17 cell-like function of IL-17RB positive NKT cells, as well as a method of preventing or treating non-allergic airway inflammation and/or non-allergic airway hyperreactivity, comprising administering an effective amount of a substance capable of inhibiting or eliminating Th17 cell-like function of IL-17RB positive NKT cells to a mammal. Examples of the substance inhibiting or eliminating the Th17 cell-like function of the IL-17RB positive NKT cells include an antagonistic antibody and a low-molecular inhibitor to IL-17RB or IL-23R, an antibody to IL-25 or IL-23, and a soluble molecule of IL-17RB or IL-23R. Further, examples include such a substance that inhibits expression of IL-17RB or IL-23R in the NKT cells, for example, an antisense nucleic acid (e.g. a DNA, an RNA, or a modified nucleotide, or a chimeric molecule thereof), a ribozyme, an RNAi-inducing nucleic acid (a polynucleotide capable of inducing the RNAi effect by introduction into a cell, preferably RNA: e.g. siRNA), an aptamer, and an expression vector comprising a nucleic acid encoding them.

Examples of the antagonistic antibody to IL-17RB include antibodies which antagonistically act on IL-17RB, among the aforementioned antibodies used for detecting and separating the IL-17RB positive NKT cells. Confirmation of the action can be performed by investigating an influence of the IL-17RB positive NKT cells induced by ligand stimulation, on the Th17 cell-like function. As the low-molecular inhibitor, a low-molecular inhibitor screened by using an IL-17RB overexpressing cells (e.g. IL-17RB overexpressing 293T cells), or measuring binding property to a soluble recombinant can be used. The antibody to IL-23R, IL-25 and IL-23 used in the present invention can be prepared as a polyclonal antibody or a monoclonal antibody thereof, by the well-known immunological procedure, as in the antibody to IL-17RB. Alternatively, the antibody may be a fragment of an antibody (e.g. Fab, F(ab′)₂), or a recombinant antibody (e.g. single strand antibody). As the substance capable of inhibiting or eliminating the Th17 cell-function of the IL-17RB positive NKT cells, an IL-17RB soluble molecule can be used. The IL-17RB soluble molecule is a protein molecule containing a partial amino acid sequence Including an extracellular region of IL-17RB. Specifically, IL-17RB is a protein molecule having an amino acid sequence shown by SEQ ID NO: 67, wherein the 1-position-17-position is considered to be a signal sequence, the 18-position-289-position is considered to be an extracellular region, the 290-position-313-position is considered to be a transmembrane region, and thereafter is considered to be an intracellular domain. IL-23R soluble molecule is a protein molecule containing a partial amino acid sequence containing the extracellular region of IL-23R. Specifically, IL-23R is a protein molecule having an amino acid sequence shown by SEQ ID NO: 68, wherein the 1-position-23-position is considered to be a signal sequence, the 24-position-355-position is considered to be an extracellular region, the 356-position-376-position is considered to be a transmembrane region, and thereafter is considered to be an intracellular domain.

When the substance capable of inhibiting or eliminating the Th17 cell-like function of IL-17RB positive NKT cells, which is an active component, is a nucleic acid molecule or a protein molecule, the therapeutic agent of the present invention can also contain an expression vector comprising the nucleic acid molecule or a nucleic acid molecule encoding the protein molecule as an active component. The expression vector must be such that an oligonucleotide or a polynucleotide encoding the nucleic acid molecule is functionally connected to a promoter capable of exerting the promoter activity in a cell of a mammal to which the expression vector is administered. The promoter which can be contained in the expression vector of the present invention is not particularly limited, as far as it enables expression of a factor under its control, but is arbitrarily selected depending on a kind of the factor, and examples thereof include a polIII promoter (e.g. tRNA promoter, U6 promoter, H1 promoter), and a promoter for a mammal (e.g. CMV promoter, CAG promoter, SV40 promoter). Alternatively, as the promoter to be used, a promoter specific for a lymphocyte (e.g. lck promoter, Pmed1 promoter) may be used.

The expression vector preferably contains a transcription termination signal, that is, a terminator region downstream of an oligo(poly)nucleotide encoding a nucleic acid molecule. Further, the expression vector can further contain a selection marker gene for selecting transformed cells (a gene which imparts resistance to a drug such as tetracycline, ampicillin, kanamycin, hygromycin, phosphinothricin etc., gene for supplementing an auxotrophy mutation etc.)

A vector of a fundamental skeleton used as the expression vector may be a plasmid or a virus vector, and examples of a vector suitable for administration to a mammal such as a human etc. include virus vectors such as adenovirus, retrovirus, adeno-associated virus, herpes virus, vaccinia virus, poxvirus, poliovirus, sindbis virus, sendai virus etc.

The therapeutic agent of the present invention can contain an arbitrary carrier, for example, a pharmaceutically acceptable carrier, in addition to the substance capable of inhibiting or eliminating the Th17 cell-like function of the IL-17RB positive NKT cells. Examples of the pharmaceutically acceptable carrier are not limited to, but include excipients such as sucrose, starch, mannit, sorbit, lactose, glucose, cellulose, talc, calcium phosphate, calcium carbonate etc., binders such as cellulose, methylcellulose, hydroxypropylcellulose, polypropylpyrrolidone, gelatin, gum arabic, polyethylene glycol, sucrose, starch etc., disintegrating agents such as starch, carboxymethylcellulose, hydroxypropylstarch, sodium-glycol-starch, sodium bicarbonate, calcium phosphate, calcium citrate, etc., lubricants such as magnesium stearate, aerosil, talc, sodium laurylsulfate etc., aromatic substances such as citric acid, menthol, glycyrrhizin.ammonium salt, glycine, orange powder etc., preservatives such as sodium benzoate, sodium hydrogen sulfide, methylparaben, propylparaben etc., stabilizers such as citric acid, sodium citrate, acetic acid etc., suspending agents such as methylcellulose, polyvinylpyrrolidone, aluminum stearate etc., dispersants such as surfactant etc., diluents such as water, physiological saline, orange juice etc., base waxes such as cacao butter, polyethylene glycol, kerosene etc.

Preparations suitable for oral administration are liquid preparations in which an effective amount of a substance is dissolved in a diluting solution such as water and physiological saline, capsules, saches or tablets containing an effective amounts of a substance as a solid or a granule, suspensions in which an effective amount of a substance is suspended in a suitable dispersing medium, emulsions in which a solution with an effective amount of a substance dissolved therein is dispersed and emulsified in a suitable dispersing medium, powders, granules etc.

As preparations suitable for parenteral administration (e.g. intravenous injection, subcutaneous injection, intramuscular injection, local injection etc.), there are aqueous and non-aqueous isotonic sterile injection liquid preparations, and these may contain antioxidants, buffers, bacteriostatic agents, tonicity agent etc. In addition, the examples include aqueous and non-aqueous sterile suspensions, and these may contain suspending agent, solubilizers, thickeners, stabilizers, antiseptics etc. The preparation can be sealed into a container by a unit dose or a plurality of doses, like ampoules or vials. Alternatively, an active ingredient and a pharmaceutical acceptable carrier are lyophilized, and may be stored in the state where it may be dissolved or suspended in a suitable sterile vehicle immediately before use.

An administration method and a dosage form of the substance capable of inhibiting or eliminating the Th17 cell-like function of IL-17RB positive NKT cells, which is an active component of the therapeutic agent of the present invention, are not particularly limited, but are intravenous administration, intra-arterial administration, intramuscular administration, oral administration, suppository administration etc., and the substance can be formulated into oral or parenteral administration by combining with pharmaceutically acceptable excipients or diluents. Administration is performed once or by dividing into several times per day, and an amount of dose is determined depending on the conditions such as severity, age, sex, weight etc. of a patient, and is in such a range that the side effect is not generated.

### EXAMPLES

The present invention will be explained in detail below by way of Examples, but these Examples do not limit a scope of the present invention at all. In addition, reagents, devices and materials used in the present invention are commercially available, unless otherwise is indicated.

### Material and Method

### (1. Targeting)

Genome fragments designed as a long arm and a short arm were subcloned from BAC clone (RP23-234M9) containing mouse IL-17RB gene locus, and exon2 of IL-17RB gene was replaced with neomycin gene to perform gene destruction.

### (2. FACS Analysis)

The cells were stained with the following antibodies, and analyzed by FACSCalibur (manufactured by BD Biosciences) or FACSCantoII (manufactured by BD Biosciences). The cells were sorted and, when subjected to in vitro assay or transfer experiment, purified by FACSAria (manufactured by BD Biosciences). The antibodies (manufactured by BD Biosciences) used were as follows.

FITC or APC-Cy7 anti-TCRβ (H57-597)
Pacific blue anti-CD4 (RM4-5)
FITC anti-CD44 (IM7)
PE-Cy7 anti-NK1.1 (PK136)
PE anti-CD122 (TM-β1)
FITC anti-CD8a (53-6.7)
PerCP-Cy5.5 anti-CD25 (PC61).

### (3. Analysis of Cytokine Production)

### 3.1 Measured at Protein Level

For measurement of cytokine in culture supernatant, IL-22 was quantified by ELISA kit (manufactured by eBioscience). Other cytokines were quantitatively analyzed by cytokine beads array method (manufactured by BD Biosciences).

3.2 Measured at mRNA Level)

Quantitative PCR was performed by TagMan method (manufactured by Applied Biosystems) using ROX as a reference, and analyzed by ABI PRISM7900HT (manufactured by Applied Biosystems) or Biomark (manufactured by Fludigm). The primers and probes used for analyzing the expression level of each gene were as follows.

TABLE 1

| Gene | | Sequence |
|---|---|---|
| Cd4 | Forward | CTGACTCTGACTCTGGACAAAGG |
| | primer | (SEQ ID NO: 1) |
| | Reverse | GGAGAGGTAGGTCCCATCACC |
| | primer | (SEQ ID NO: 2) |
| | Probe | TTGAGCTGAGCCACTTTCATCACCACCA |
| | | (SEQ ID NO: 3) |
| Il17rb | Forward | CCAGATGACAACAGACGCATG |
| | primer | (SEQ ID NO: 4) |
| | Reverse | GAGCATGGTGGAAATAGGAAAGG |
| | primer | (SEQ ID NO: 5) |
| | Probe | CGTCTTCGTGCTCCTTCCTTGCCTCC |
| | | (SEQ ID NO: 6) |
| Il2rb | Forward | GAAGGGTTGGCGTAGGGTAAAG |
| | primer | (SEQ ID NO: 7) |
| | Reverse | GCAGAACTTGGAGGGAATGAGG |
| | primer | (SEQ ID NO: 8) |
| | Probe | TCCCTTTGACAACCTTCGCCTGGTGG |
| | | (SEQ ID NO: 9) |
| Ifng | Forward | GGATGCATTCATGAGTATTGCCAAG |
| | primer | (SEQ ID NO: 10) |
| | Reverse | CTCCTTTTCCGCTTCCTGAGG |
| | primer | (SEQ ID NO: 11) |
| | Probe | AGGTCAACAACCCACAGGTCCAGCG |
| | | (SEQ ID NO: 12) |
| Tbx21 | Forward | AAGGATTCCGGGAGAACTTTGAG |
| | primer | (SEQ ID NO: 13) |
| | Reverse | TGGTTGGATAGAAGAGGTGAGAAG |
| | primer | (SEQ ID NO: 14) |
| | Probe | TGTACGCATCTGTTGATACGAGTGTCCCCT |
| | | (SEQ ID NO: 15) |
| Stat4 | Forward | AGGGAAGAGAGGAGAATATTGGC |
| | primer | (SEQ ID NO: 16) |
| | Reverse | GTTCCACATTCCTTTGTCTTTCAG |
| | primer | (SEQ ID NO: 17) |
| | Probe | CAGCCAACATGCCTATCCAGGGACCT |
| | | (SEQ ID NO: 18) |
| Il4 | Forward | CATCGGCATTTTGAACGAGGTC |
| | primer | (SEQ ID NO: 19) |
| | Reverse | CGTTGCTGTGAGGACGTTTG |
| | primer | (SEQ ID NO: 20) |
| | Probe | TCTCCGTGCATGGCGTCCCTTCTCC |
| | | (SEQ ID NO: 21) |
| Gata3 | Forward | GCTACGGTGCAGAGGTATCC |
| | primer | (SEQ ID NO: 22) |
| | Reverse | TCCAGCCAGGGCAGAGATC |
| | primer | (SEQ ID NO: 23) |
| | Probe | CGACCCACCACGGGAGCCAGGT |
| | | (SEQ ID NO: 24) |
| Il17a | Forward | CCTTGGCGCAAAAGTGAGC |
| | primer | (SEQ ID NO: 25) |
| | Reverse | ATATCTATCAGGGTCTTCATTGCG |
| | primer | (SEQ ID NO: 26) |
| | Probe | ACTACCTCAACCGTTCCACGTCACCC |
| | | (SEQ ID NO: 27) |
| Il22 | Forward | AGCTTGAGGTGTCCAACTTCC |
| | primer | (SEQ ID NO: 28) |
| | Reverse | AACAGTTTCTCCCCGATGAGC |
| | primer | (SEQ ID NO: 29) |
| | Probe | AGCCGTACATCGTCAACCGCACCT |
| | | (SEQ ID NO: 30) |
| Rorc | Forward | GGCTTTCCATCATCATCTCTGC |
| | primer | (SEQ ID NO: 31) |
| | Reverse | GGTGGAGGTGCTGGAAGATC |
| | primer | (SEQ ID NO: 32) |
| | Probe | CCTCCTAGCCAAGCTGCCACCCAAAG |
| | | (SEQ ID NO: 33) |
| Ill7ra | Forward | GTGCCCTGCCCAGTAATCTC |
| | primer | (SEQ ID NO: 34) |
| | Reverse | ATGGCGATGAGTGTGATGAGG |
| | primer | (SEQ ID NO: 35) |
| | Probe | ACCACAGTTCCCAAGCCAGTTGCAGA |
| | | (SEQ ID NO: 36) |
| Il12rb1 | Forward | CGCTGCGAGGCTGAAGAC |
| | primer | (SEQ ID NO: 37) |
| | Reverse | CGCAGTCCGTCAAGTGTCAC |
| | primer | (SEQ ID NO: 38) |
| | Probe | CACGAGCCACTCTGACTCCCACGC |
| | | (SEQ ID NO: 39) |
| Il12rb2 | Forward | CGCTTCTGCACCCACTCAC |
| | primer | (SEQ ID NO: 40) |
| | Reverse | TGCCAGGTCACTAGAATGTTGTC |
| | primer | (SEQ ID NO: 41) |
| | Probe | CACTGGGTTGCTGGCTCCTCACCA |
| | | (SEQ ID NO: 42) |
| I123r | Forward | GCTTCTACTACATTTGGGACATGAG |
| | primer | (SEQ ID NO: 43) |
| | Reverse | CACCAGGCTCAACCCACATG |
| | primer | (SEQ ID NO: 44) |
| | Probe | TGATTCCTCCGTGACACCATCTGAAGAGCA |
| | | (SEQ ID NO: 45) |
| Ccr4 | Forward | CTCAGGATCACTTTCAGAAGAGC |
| | primer | (SEQ ID NO: 46) |
| | Reverse | GGTGGTGTCTGTGACCTCTG |
| | primer | (SEQ ID NO: 47) |
| | Probe | AGGCAGCTCAACTGTTCTCATTGGCT |
| | | (SEQ ID NO: 48) |
| Ccr6 | Forward | CTGCCCACTTCCCTTTCTACAC |
| | primer | (SEQ ID NO: 49) |
| | Reverse | CTGTGTTGTCATAATCATCCGTTCC |
| | primer | (SEQ ID NO: 50) |
| | Probe | TCATTCCCCAGGCAGGCGTGGTTCT |
| | | (SEQ ID NO: 51) |
| Ccr7 | Forward | CATGGACCCAGGGAAACCC |
| | primer | (SEQ ID NO: 52) |
| | Reverse | TGACCTCATCTTGGCAGAAGC |
| | primer | (SEQ ID NO: 53) |
| | Probe | TGACAAGGAGAGCCACCACCAGCACG |
| | | (SEQ ID NO: 54) |
| Cxcr3 | Forward | GAAGCAGGCAGCACGAGAC |
| | primer | (SEQ ID NO: 55) |
| | Reverse | CCGAGGCATCTAGCACTTGAC |
| | primer | (SEQ ID NO: 56) |
| | Probe | CGGAGCACCAGCCAAGCCATGTACC |
| | | (SEQ ID NO: 57) |
| Cxcr6 | Forward | CACACTTCACTCTGGAACAAAGC |
| | primer | (SEQ ID NO: 58) |
| | Reverse | TGGCTGTTATCACTGGAATTGTTG |
| | primer | (SEQ ID NO: 59) |
| | Probe | AGCCAGAAATCTCCCTCGTAGTGCCCATC |
| | | (SEQ ID NO: 60) |
| E44p4 | Forward | ACAGCCGCCCTTTCTTTTCC |
| | primer | (SEQ ID NO: 61) |
| | Reverse | GGACTTCAGCCTCTCATCCATC |
| | primer | (SEQ ID NO: 62) |
| | Probe | ACCAGGGAGCAGAACCACGATAACCC |
| | | (SEQ ID NO: 63) |
| Hprt | Forward | GAGGATTTGGAAAAAGTGTTTATTCCTC |
| | primer | (SEQ ID NO: 64) |
| | Reverse | GATGGCCTCCCATCTCCTTC |
| | | (SEQ ID NO: 65) |
| | Probe | CATCTCGAGCAAGTCTTTCAGTCCTGTCCA |
| | | (SEQ ID NO: 66) |

### (4. Co-Culture of Dendritic Cells and NKT Cells)

Mouse bone marrow was collected, mononuclear cells obtained after hemolysis were plated for 1-1.5 hr on a plate immobilized with human IgG and matured cells were removed by adsorption. Immature mononuclear cells were cultured at 5×10⁵/mL in the presence of GM-CSF (10 ng/mL) for 5 or 6 days to induce differentiation into bone marrow-derived dendritic cells (BM-DCs). NKT cells or NKT cell subgroup (5×10⁴) purified by sorting were co-cultured with 5×10³ BM-DCs. Under the present conditions, 1-100 ng/mL α-GalCer or 0.1-10 ng/mL cytokine (IL-12, IL-23, IL-25) was added during culture to stimulate NKT cells, and cell supernatant and cells at 24-72 hr from the start of the culture were subjected to experiments.

### (5. Analysis of Gene Expression Profile by DNA Microarray)

Total RNA was extracted from the cells, and the gene expression level was comprehensively analyzed by a hybridization method of cRNA using GeneChip (manufactured by Affimetrix). The correlation based on Pearson distribution was analyzed by clustering analysis.

### (6. Analysis of Infiltrated Cells in Bronchoalveolar Washing)

The infiltrated cells in the bronchoalveolar washing were subjected to FACS analysis, and F4/80 positive was identified as macrophages, CCR3 positive was identified as kousankyu, Gr1 positive was identified as neutrophils and CD45 positive was identified as lymphocytes.

### Example 1

### Preparation of IL-17RB Deficient Mice and Evaluation of NKT Cells

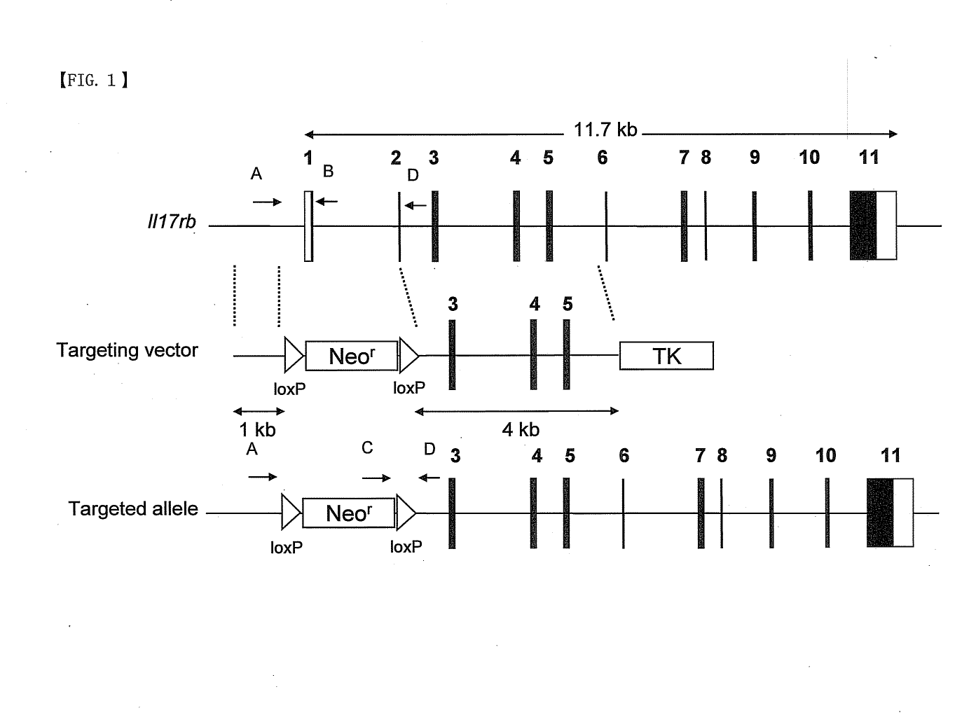

With regard to IL-17RB gene showing NKT cell subgroup-specific expression in a steady state, a targeting vector was constructed such that exon1 and exon2 on the mouse genome were deficient (FIG. 1), and gene targeting in ES cells was performed. Insertion into an object site was confirmed by genomic PCR. The mouse line was established by mating C57BL/6 or Balb/c mice for 8 generations or more to finally establish IL-17RB deficient mice of C57BL/6 or Balb/c background.

As IL-15 mutant mice (IL-15^{L117P} mice), the mice established by chemical mutagen ENU (N-ethyl-N-nitrosourea)-induced mutation (Masuya H, et al. (2004) Development and implementation of a database system to manage a large-scale mice ENU-mutagenesis program. Mamm Genome 15: 404-411 (2004); Yoshida Y, et al. (2009) PosMed (Positional Medline): prioritizing genes with an artificial neural network comprising medical documents to accelerate positional cloning. Nucleic Acids Res. 37: W147-W152.) were used.

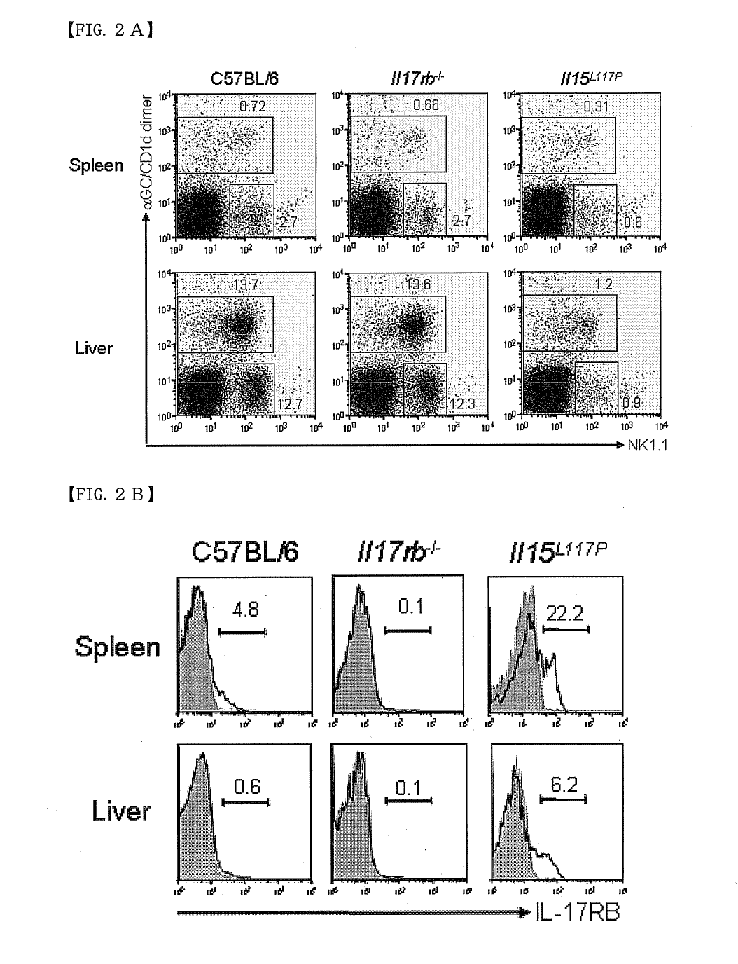

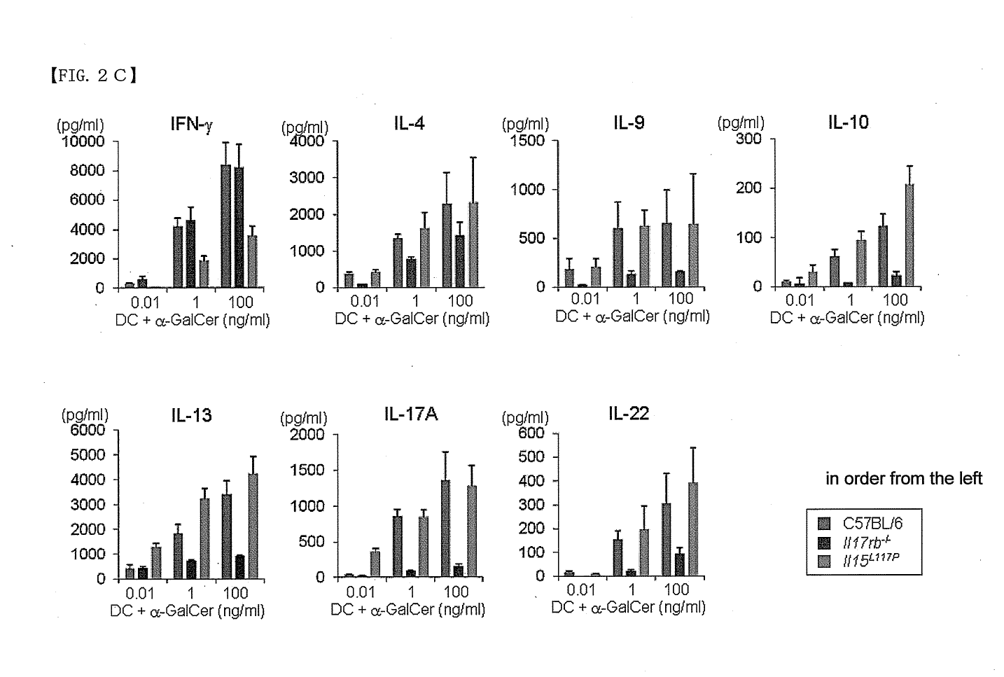

C57BL/6 background IL-17RB deficient mice-derived NKT cells showed a certain decrease of cell number in the spleen and liver, though not to the level of decrease of the number of NK cells and NKT cells in IL-15 mutant mice (IL-15^{L117P} mice) (FIG. 2A).

On the contrary, the proportion of IL-17RB positive NKT cells increased in IL-15 mutant mice, and therefore, IL-17RB positive NKT cells were assumed to have not IL-15 requirement (FIG. 2B).

The mechanism in cytokine production of NKT cells has heretofore been known to recognize glycolipid presented to a CD1d molecule on an antigen presenting cell, and produce many cytokines. In fact, it is known that IFN-γ, IL-4, IL-17A and the like are produced by co-culture NKT cells with bone marrow-derived dendritic cell (DC; GM-CSF induction) in vitro in the presence of glycolipid ligand α-galactosylceramide (α-GalCer). Cytokine production by IL-17RB deficient mice splenic NKT cells was examined.

As a result, as compared to wild-type mice splenic NKT cells, no abnormality was found in IFN-γ production but the production of IL-4, IL-9, IL-10, IL-13, IL-17A, IL-22 markedly decreased (FIG. 2C). This tendency was the same as with liver NKT cells.

On the other hand, in IL-15^{L117P} mice, although IFN-γ production decreased markedly, no abnormality was found in the production of IL-4, IL-9, IL-10, IL-13, IL-17A, IL-22 (FIG. 2C).

The same tendency as in these results was also found in blood cytokine production by intravenous injection of α-GalCer.

### Example 2

### Mechanism of Occurrence of Differentiation of IL-17RB Positive NKT Cells

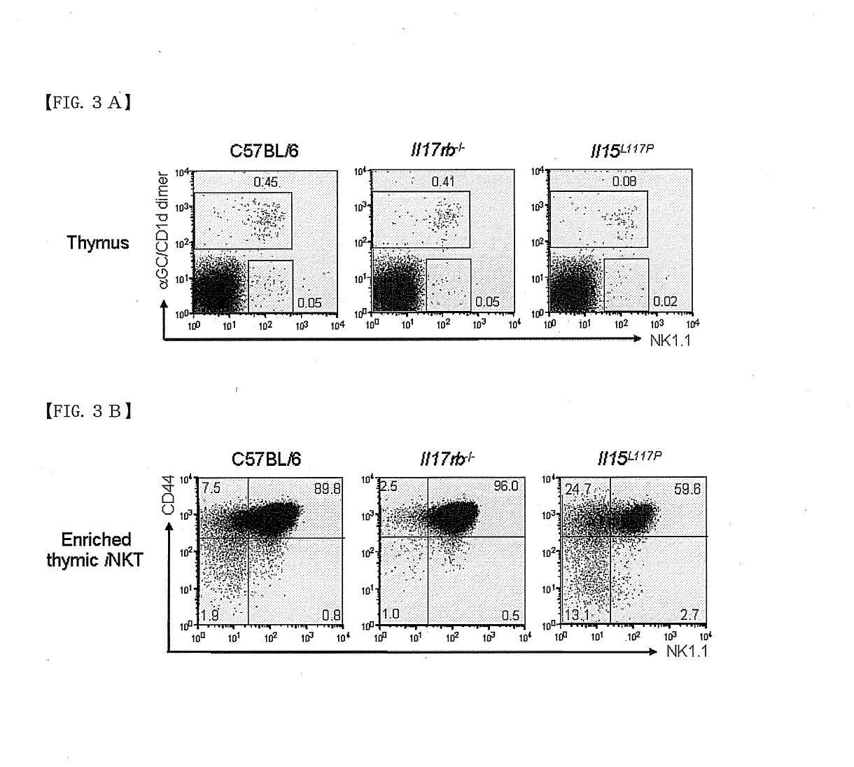

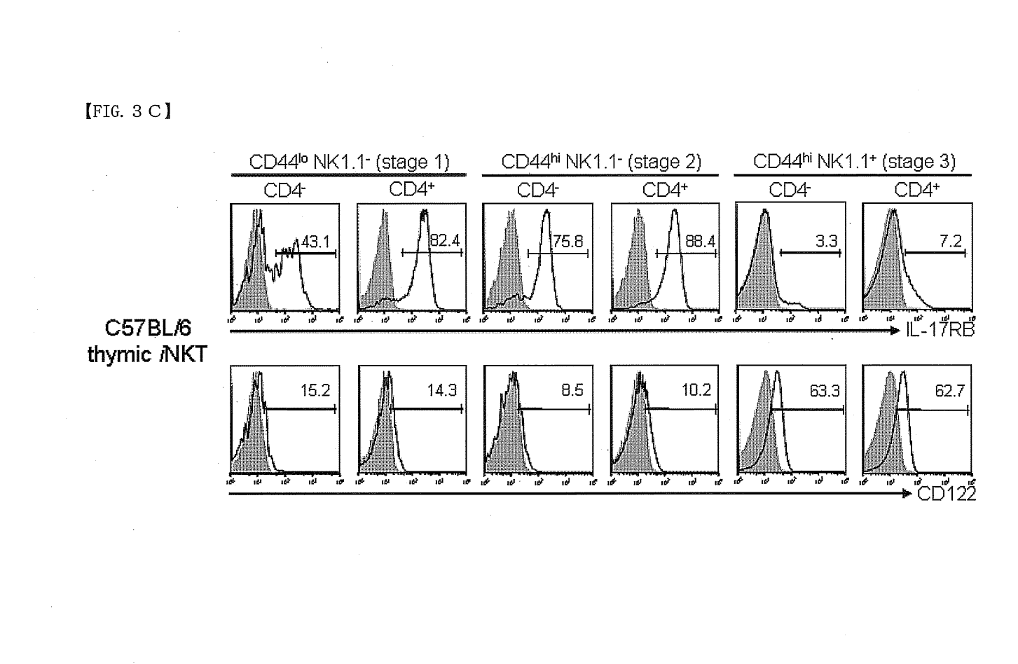

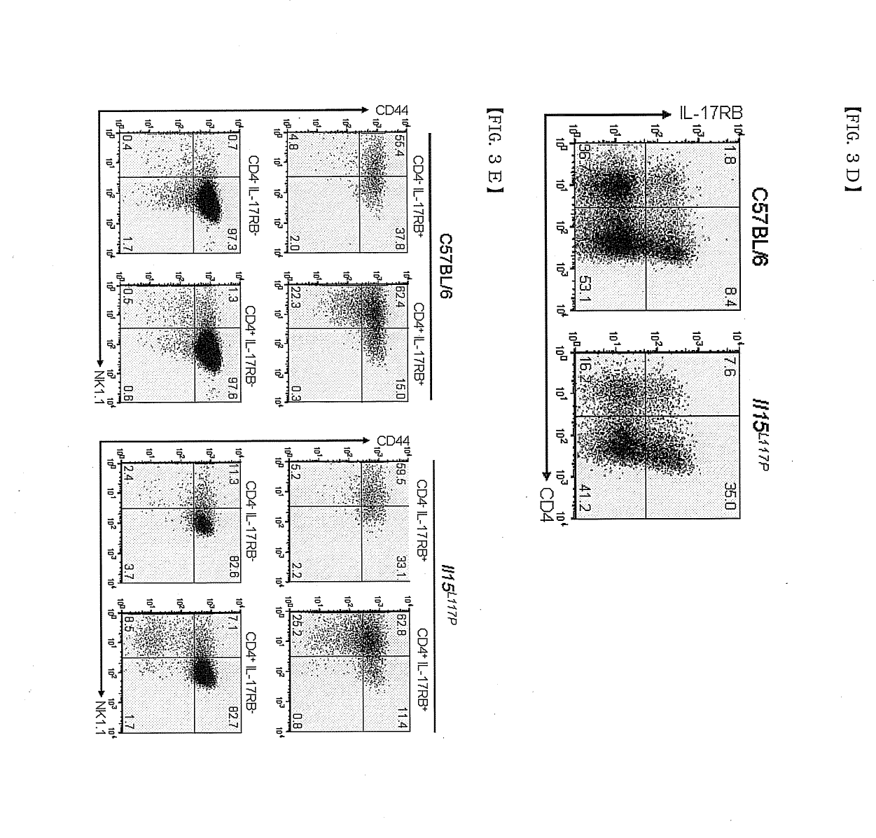

NKT cells are known to differentiate in the thymus same as T cells. To clarify whether the subtype group IL-17RB positive NKT cells also differentiate in the thymus (or differentiate in the periphery), NKT cells in the thymus were analyzed. Like the peripheral NKT cells described in Example 1, the proportion of thymic NKT cells scarcely decreased (FIG. 3A). NKT cells were concentrated with MACS beads, and the expression state of NK1.1 and CD44 was confirmed. As a result, in wild-type mice, 3 stages of thymic differentiation considered so far, that is, CD44 negative NK1.1 negative (stage 1), CD44 positive NK1.1 negative (stage 2), and CD44 positive NK1.1 positive (stage 3) could be confirmed, whereas the proportion of stages 1 and 2 decreased in IL-17RB deficient mice, and the proportion of stage 3 decreased in IL-15 mutant mice (FIG. 3B). Therefore, since an inversely-correlated cell decrease is found in IL-17RB deficient mice m and IL-15 mutant mice, and the differentiation stages are unlikely in a cascade of stage 1-+2-*3, expression of IL-17RB and CD122 (IL-15 receptor β chain) in each stage was confirmed. As a result, localization of IL-17RB expression in stages 1 and 2, and localization of CD122 expression in stage 3 were clarified (FIG. 3C), thus confirming correlation to the results of FIG. 3B.

In IL-15 mutant mice, the proportion of IL-17RB positive cells in NKT cells increased and the number did not change (FIG. 3D).

In wild-type mice, CD4 negative IL-17RB positive NKT cells were localized in stage 2, CD4 positive IL-17RB positive NKT cells were localized in stages 2 and 1, and IL-17RB negative NKT cells were localized in stage 3 (FIG. 3E). On the other hand, in IL-15 mutant mice, although IL-17RB positive NKT cells did not change, the number of IL-17RB negative NKT cells decreased and cells in stage 1 increased in CD4 positive IL-17RB negative NKT cells (FIG. 3E).

In summary fashion, these results have clarified that not less than 80% of stages 1 and 2 were IL-17RB positive NKT cells, whereas almost all cells in stage 3 were IL-17RB negative NKT cells (FIG. 3F), IL-17RB positive NKT cells did not require IL-15, and IL-17RB negative NKT cells were IL-15 requirement (FIG. 3F).

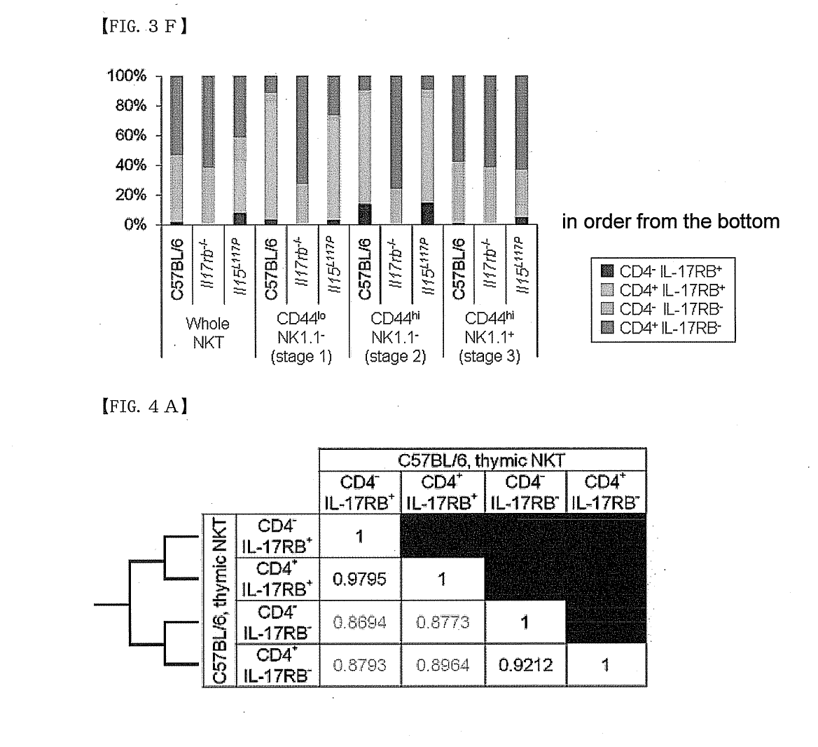

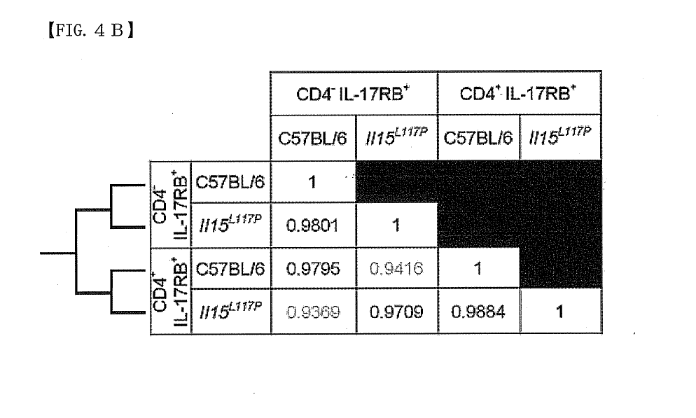

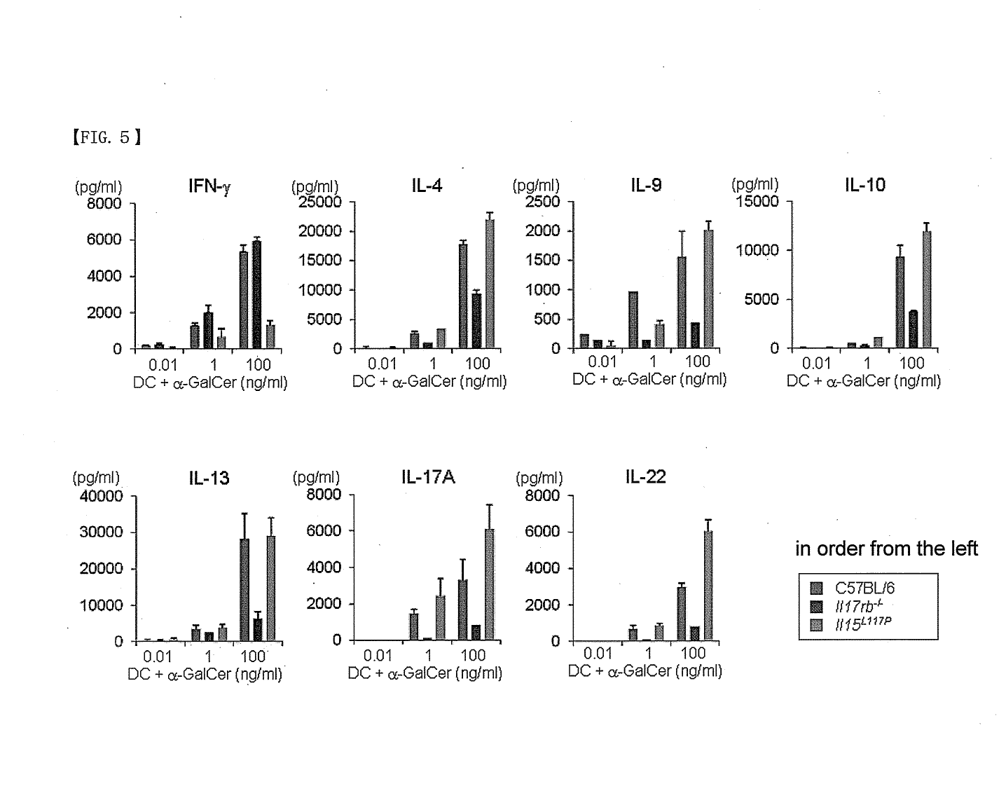

In fact, the results of gene expression profile by DNA microarray have clarified that CD4 positive IL-17RB positive NKT cells of thymus were close to CD4 negative IL-17RB positive NKT cells and far from IL-17RB negative NKT cells (FIG. 4A), CD4 negative IL-17RB positive cells derived from wild-type mice and that derived from IL-15 mutant mice are very close, and CD4 positive IL-17RB positive cells derived from wild-type mice and that derived from IL-15 mutant mice are very close (FIG. 4B). As for cytokine production by thymic NKT cells, moreover, IL-17RB deficient mice splenic NKT cells, like spleen (FIG. 2C) and liver, showed no abnormality in IFN-γ production as compared to the wild-type mice splenic NKT cells, whereas the production of IL-4, IL-9, IL-10, IL-13, IL-17A, IL-22 markedly decreased (FIG. 5). In IL-15^{L117P} mice, IFN-γ production markedly decreased but production of IL-4, IL-9, IL-10, IL-13, IL-17A, IL-22 showed no abnormality (FIG. 5).

### Example 3

### Gene Expression of Thymus NKT Cells

As indicated in Example 2, it was suggested that thymus NKT cells do not transit from stage 1→2→3, but cell populations of IL-17RB positive (mainly stages 1 and 2) and IL-17RB negative (stage 3) are separately present. To analyze in more detail the functional differences between them, the cells were divided into 4 fractions using the expression of CD4 and IL-17RB on the cell surface as an index, and the gene expression thereof was analyzed by quantitative PCR.

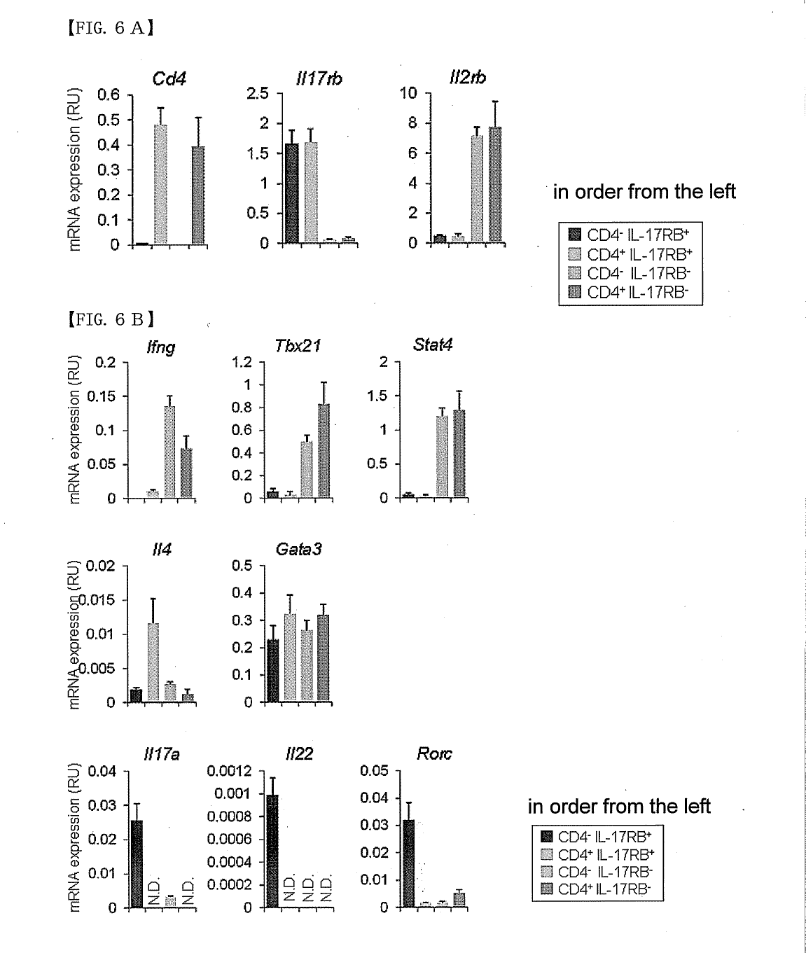

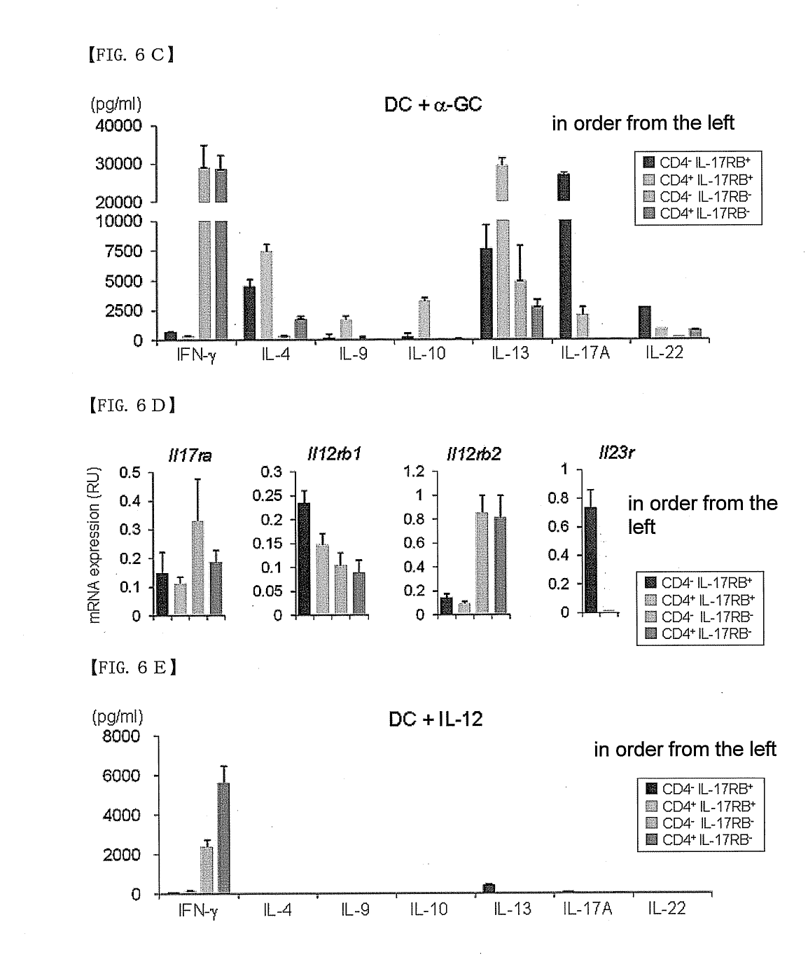

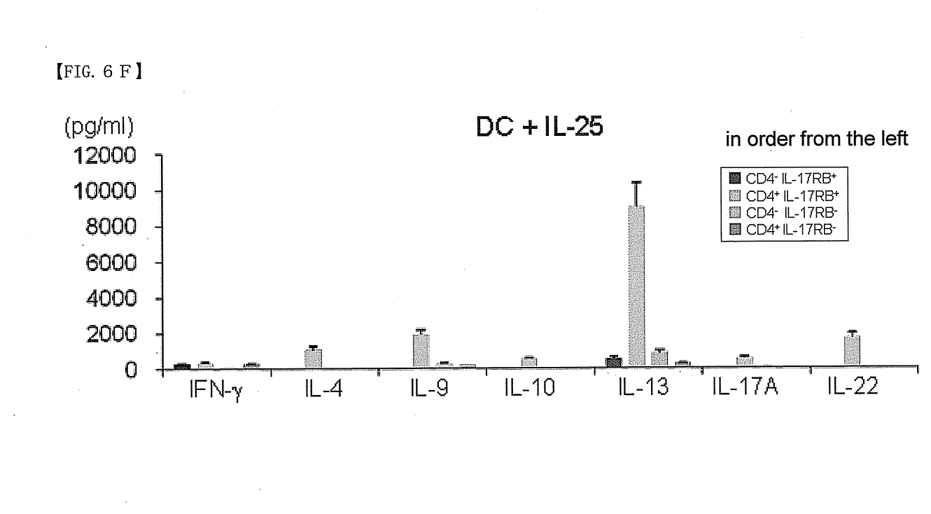

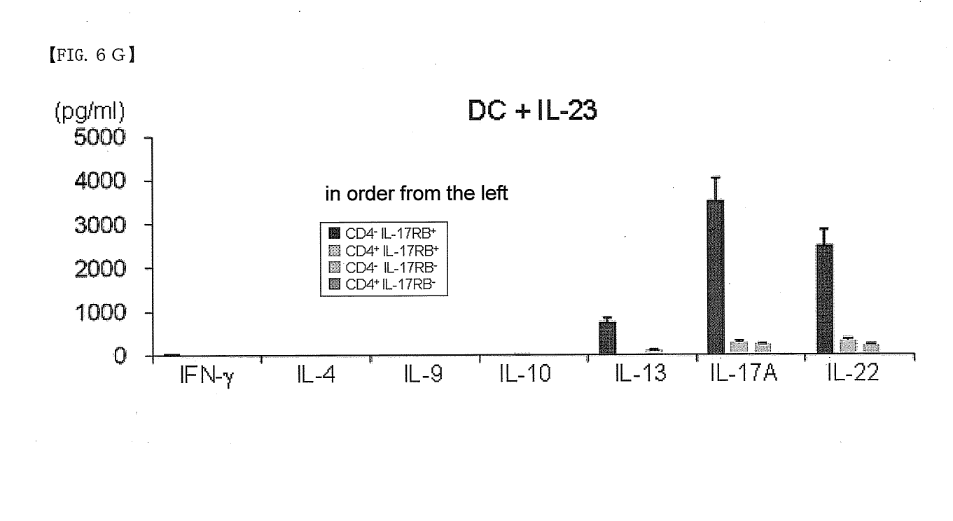

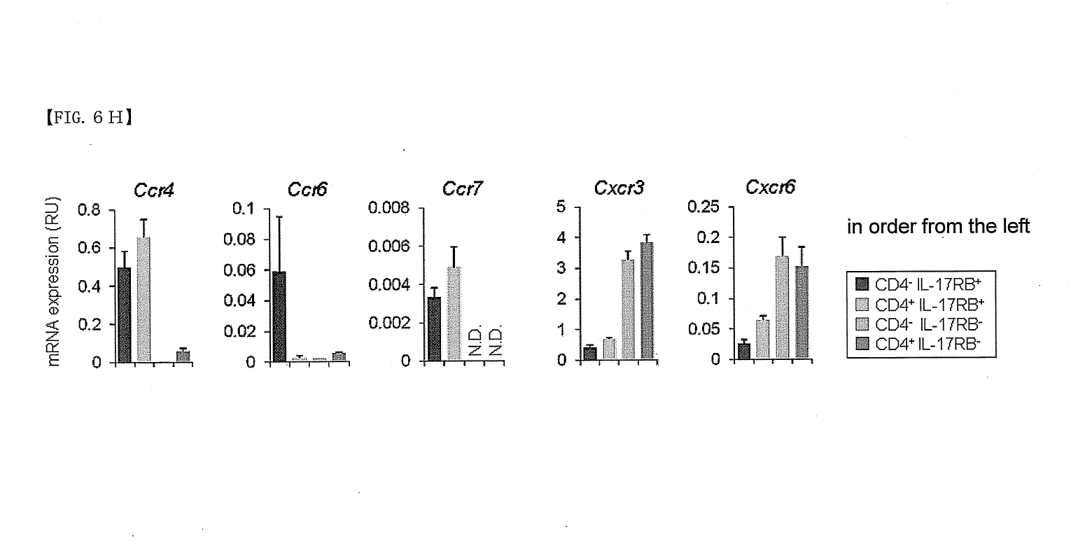

The prepared cells had high purity in view of the expression of CD4 and IL-17RB and, as clarified in Example 2, IL-15 receptor β chain (Il2rb) showed localized expression in IL-17RB negative NKT cells (FIG. 6A).

Then, Th1/Th2/Th17 cytokine production was examined.

IFN-γ (Ifng), T-bet (Tbx21), and STAT4 (Stat4), which are

Th1-related genes, showed localized expression in IL-17RB negative NKT subtype (FIG. 6B, upper panel). IL-17A(Il17a), IL-22(Il22), and RORγt (Rorc), which are Th17-related genes, showed localized expression in CD4 negative IL-17RB positive NKT subtype (FIG. 6B, lower panel). IL-4 (Il4), which is a Th2-related gene, showed high expression in CD4 positive IL-17RB positive NKT subtype, and transcription factor GATA3 (Gata3) showed expression in all NKT subtypes (FIG. 6B, middle panel). From these results, CD4 negative IL-17RB positive NKT cells, CD4 positive IL-17RB positive NKT cells, and IL-17RB negative NKT cells are considered to easily secrete Th17 cytokine, Th2 cytokine, and Th1 cytokine, respectively.

To further clarify whether each subtype reacts with a glycolipid ligand to provide a disproportionate cytokine production, each subtype was cocultured with a bone marrow-derived dendritic cell in the presence of α-GalCer in a test tube. It was confirmed IFN-γ (Th1 cytokine), IL-4, IL-9, IL-10, IL-13 (Th2 cytokines), and IL-17A, IL-22 (Th17 cytokines) are mainly produced by IL-17RB negative NKT cells, CD4 positive IL-17RB positive NKT cells (also partly produced by CD4 negative IL-17RB positive NKT cells), and CD4 negative IL-17RB positive NKT cells (also partly produced by CD4 positive IL-17RB positive NKT cells), respectively (FIG. 6C).

To confirm cytokine reactivity under physiological conditions, expression of IL-12 receptor (Il12rb2/Il12rb1), IL-23 receptor (Il123r/Il12rb1), and IL-25 receptor (Il17rb/IL17ra) was confirmed by quantitative PCR. It has been clarified that IL-12 receptor, IL-23 receptor, IL-25 receptor show localized expression in IL-17RB negative NKT cells, CD4 negative IL-17RB positive NKT cells, and IL-17RB positive NKT cells, respectively (FIG. 6D). To confirm cytokine reactivity of each subtype, each subtype was cocultured with a bone marrow-derived dendritic cell in the presence of IL-12 or IL-23 or IL-25, and cytokine production ability of NKT cells was confirmed. It has been clarified that IFN-γ is produced by IL-17RB negative NKT cells in the presence of IL-12 (FIG. 6E), IL-17A and IL-22 are mainly produced by CD4 negative IL-17RB positive NKT cells in the presence of IL-23 (FIG. 6G), and IL-4, IL-9, IL-10, IL-13, IL-17A, and IL-22 are mainly produced by CD4 positive IL-17RB positive NKT cells in the presence of IL-25 (FIG. 6F).

It has been further clarified that expression of chemokine receptor varies in each subtype. Since CD4 negative IL-17RB positive NKT cells, CD4 positive IL-17RB positive NKT cells, and IL-17RB negative NKT cells express Ccr4, Ccr6 and Ccr7, Ccr4 and Ccr7, and Cxcr3 and Cxcr6, respectively, expression of NKT subtype in the periphery is also expected to vary (FIG. 6H).

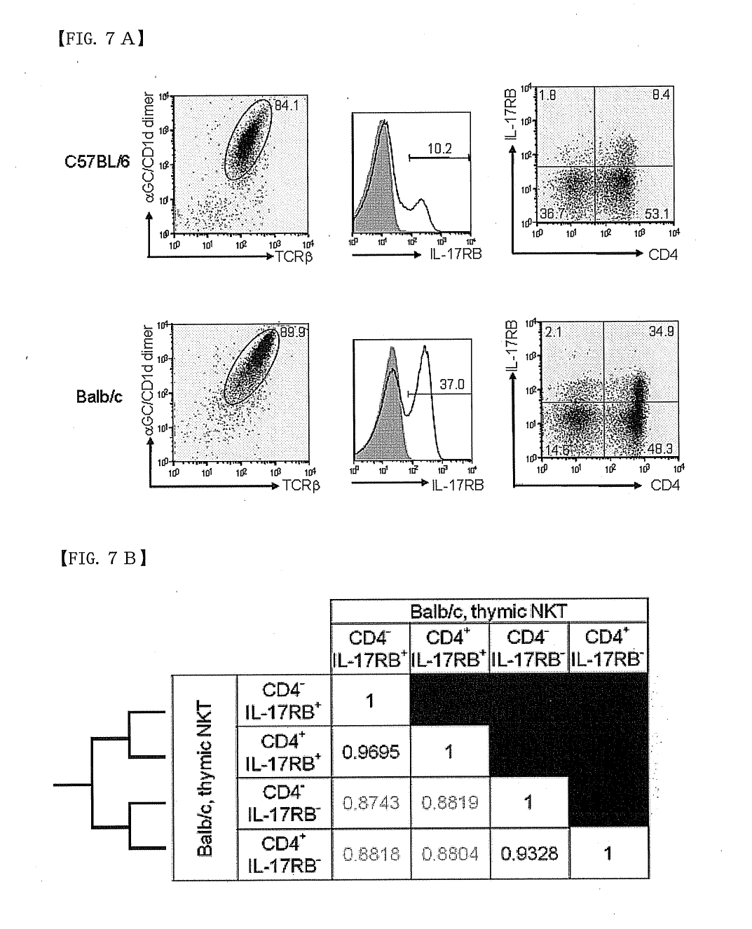

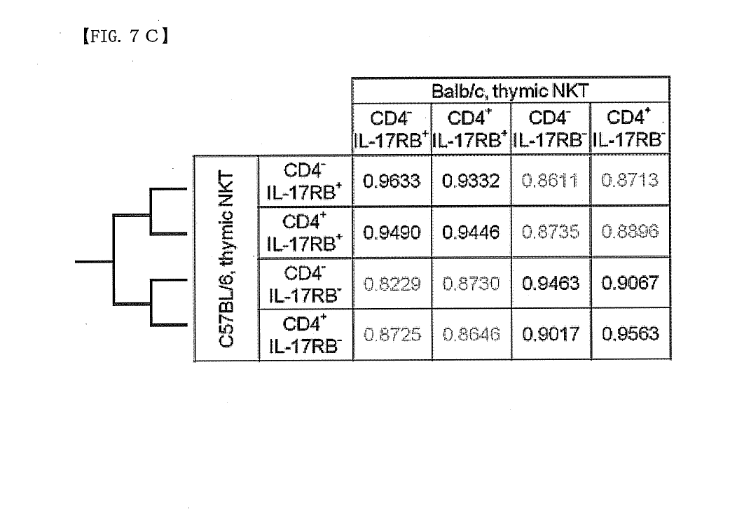

Then, whether or not the content ratio of each subtype in thymus NKT cells varies depending on the lineage of mice was examined. As a result, while the proportion of IL-17RB positive NKT cells was about 10% in C57BL/6 mice, it increased to nearly 40% in Balb/c mice. While CD4 negative IL-17RB positive was about 2% in both lineages, CD4 positive IL-17RB positive NKT cells were about 8% in C57BL/6, and about 1/3 in Balb/c (FIG. 7A).

While the proportion and number of the subtype in the both lineages were different, they were considered to be functionally equivalent. That is, the results of gene expression profile by DNA microarray have clarified that CD4 positive IL-17RB positive NKT cells of Balb/c thymus are close to CD4 negative IL-17RB positive NKT cells and far from IL-17RB negative NKT cells (FIG. 7B), CD4 negative IL-17RB positive derived from C57BL/6 and that derived from Balb/c mice are very close, and CD4 positive IL-17RB positive derived from C57BL/6 and that derived from Balb/c mice are very close (FIG. 7C). Furthermore, the expression state of IL-15 receptor β chain (Il2rb), Th1/2/17-related gene, IL-12, IL-23 and IL-25 receptors, and chemokine receptor by quantitative PCR, and cytokine production by α-GalCer, IL-12, IL-23, IL-25 response were equivalent in C57BL/6 and Balb/c.

### Example 4

### NKT Cell Subtype in Periphery

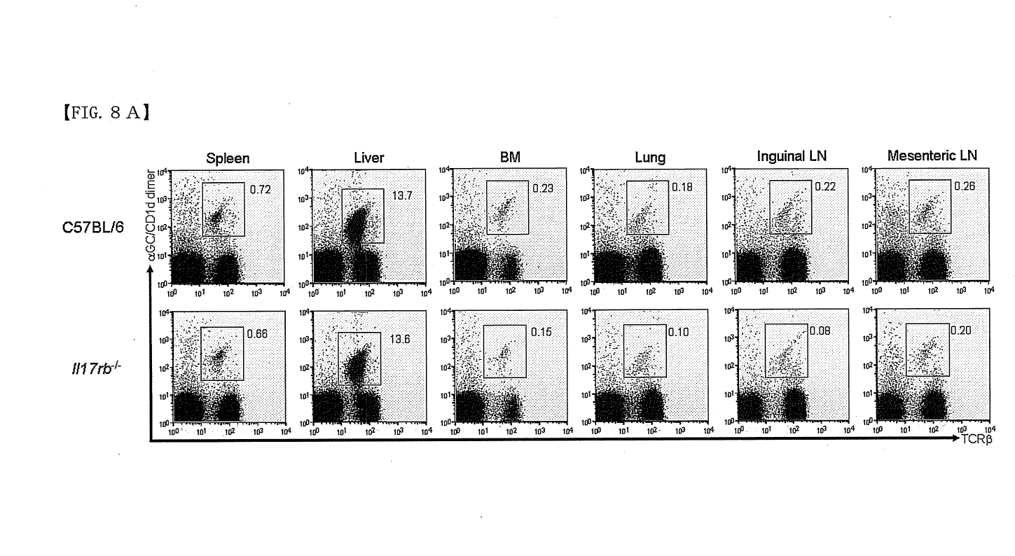

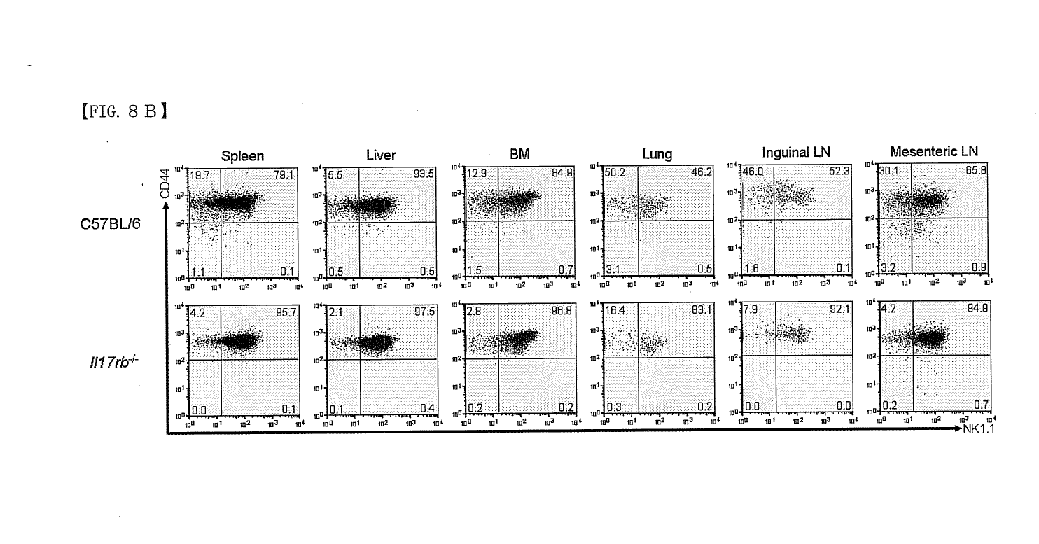

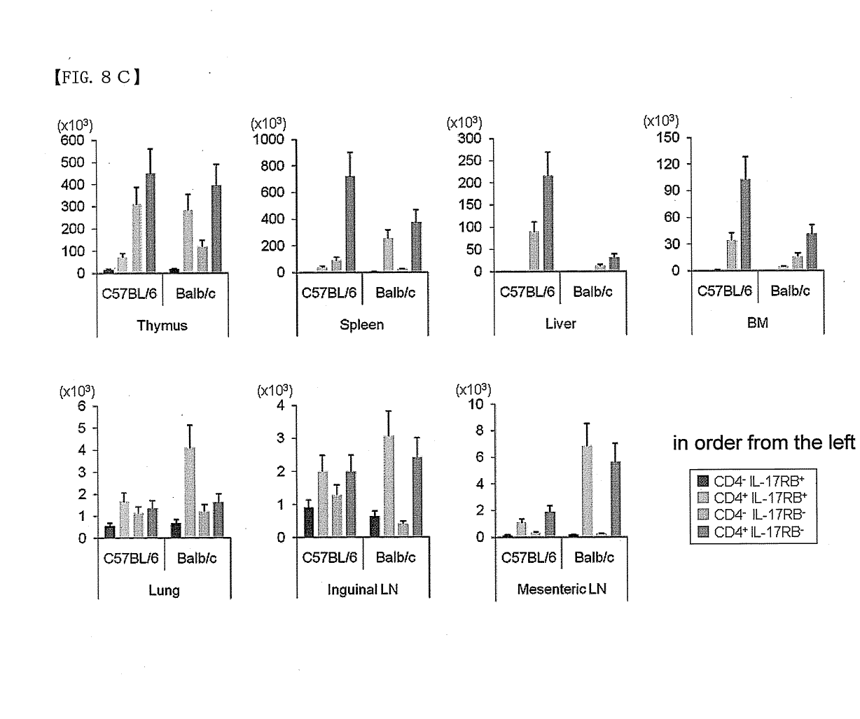

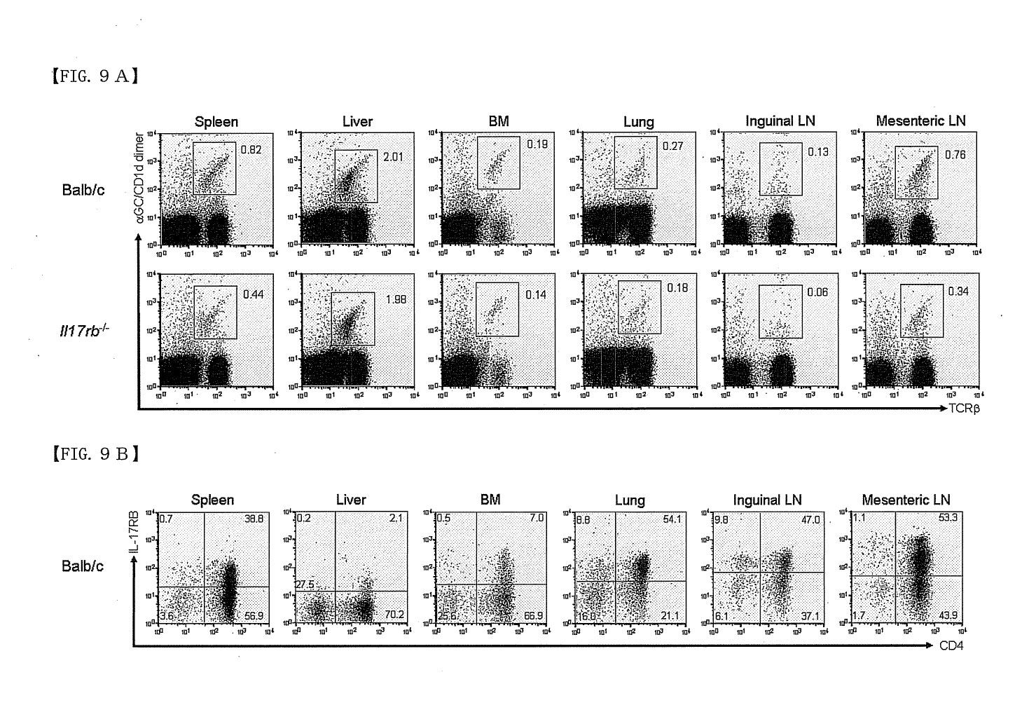

Since functionally different NKT cell subtypes are already present in the thymus, to clarify the localization of these subtypes in the periphery, NKT cells in the spleen, liver, bone marrow, lung, inguinal lymph node, and mesenteric lymph node of wild-type mice (2 lineages; C57BL/6, Balb/c) and IL-17RB deficient mice (2 lineages; C57BL/6, Balb/c) were analyzed. In IL-17RB deficient mice, both lineages did not show a remarkable decrease in the NKT cell number in the spleen, liver and bone marrow, but in the lung, inguinal lymph node and mesenteric lymph node, the number decreased to about half (FIG. 8A, FIG. 9A). In the thymus, a remarkable decrease in the populations of CD44 negative NK1.1 negative and CD44 positive NK1.1 negative was confirmed in IL-17RB deficient mice (Example 2); however, a remarkable decrease in the populations of CD44 negative NK1.1 negative and CD44 positive NK1.1 negative was similarly found in all the analyzed tissues (FIG. 8B). Thus, the proportion of 4 NKT cell subtypes divided by CD4 and IL-17RB was analyzed for each organ. Interestingly, IL-17RB positive NKT cells were scarcely present in the liver and bone marrow, whereas accumulation of CD4 negative IL-17RB positive NKT cells was found in the lung and inguinal lymph node, and accumulation of CD4 positive IL-17RB positive NKT cells was found in the spleen, lung, inguinal lymph node and mesenteric lymph node (FIG. 8C, FIG. 9B). Thus, the presence of different NKT subtypes in organs and tissues has been clarified.

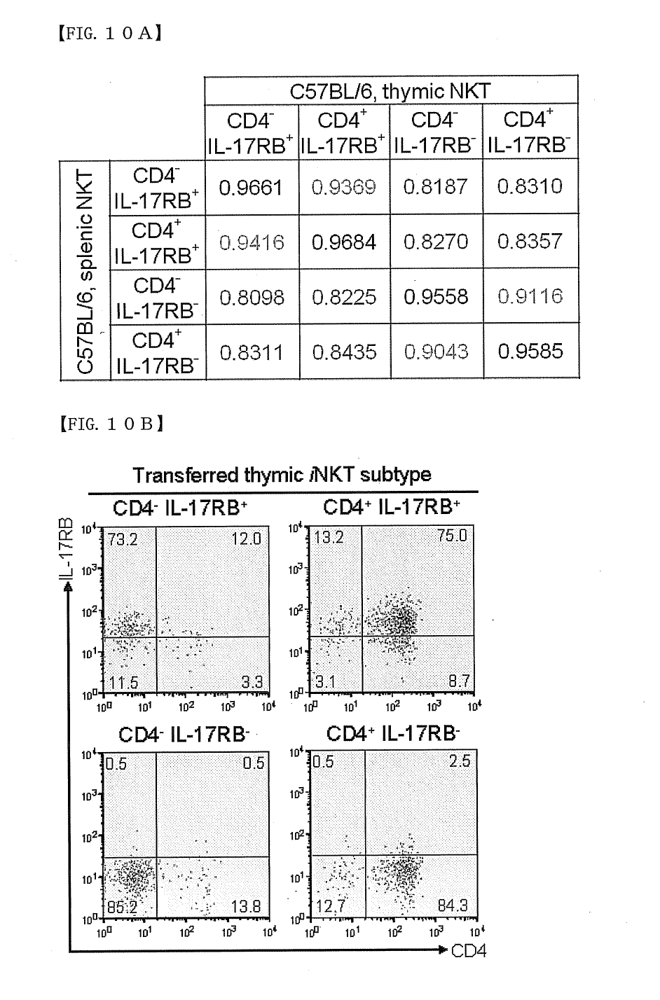

Then, whether or not each subtype of thymic NKT cells acquires plasticity after transfer to the periphery was analyzed. First, 4 subtypes derived from thymus and spleen were subjected to comprehensive gene expression analysis by DNA microarray. As a result, subtypes having the same phenotype were highly correlated, and IL-17RB positive and IL-17RB negative were far from each other (FIG. 10A). Therefore, 4 subtypes of the thymic NKT cells were sorted, each was transferred to NKT cell deficient mice (Jα18 deficient mice), and expression of CD4 and IL-17RB in NKT cells present in the spleen was confirmed one week later. As a result, it has been clarified that most of them have the same phenotypes as those before the cell transfer (FIG. 10B). Moreover, the cytokine production by each NKT cell subtype derived from the spleen by α-GalCer, IL-12, IL-23, IL-25 response showed a profile equivalent to that of thymus. It has further been clarified that the decrease of IL-17RB negative NKT cells in IL-15 mutant mice, which was also seen in the thymus, can be found in the periphery.

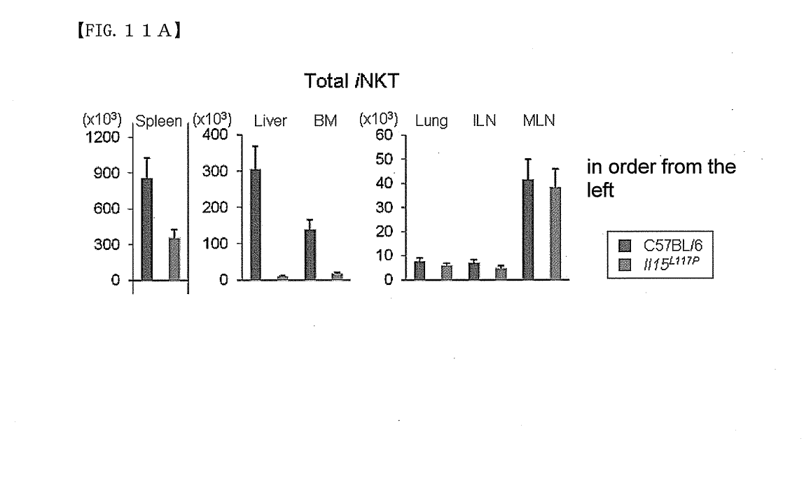

In IL-15 mutant mice, the number of NKT cells decreases to about 1/3 in the spleen and to 1/10 or below in the liver and bone marrow, whereas it scarcely changes in the lung, inguinal lymph node and mesenteric lymph node (FIG. 11A). As for each NKT subtype, the number of IL-17RB positive NKT cells does not change, but IL-17RB negative NKT cells markedly decrease in all organs, particularly liver, bone marrow and spleen (FIG. 11B). In addition, the expression profiles of the group of the series of genes subjected to the quantitative analysis in thymus in Example 3 were almost the same in all organs, and that of all genes tended to be relatively high as compared to the thymus. In fact, cytokine production by spleen NKT subtype due to α-GalCer, IL-12, IL-23, IL-25 response was equivalent to that by thymus NKT subtype.

### Example 5

### Cytokine Production Mechanism of CD4 Positive IL-17RB Positive NKT Cells

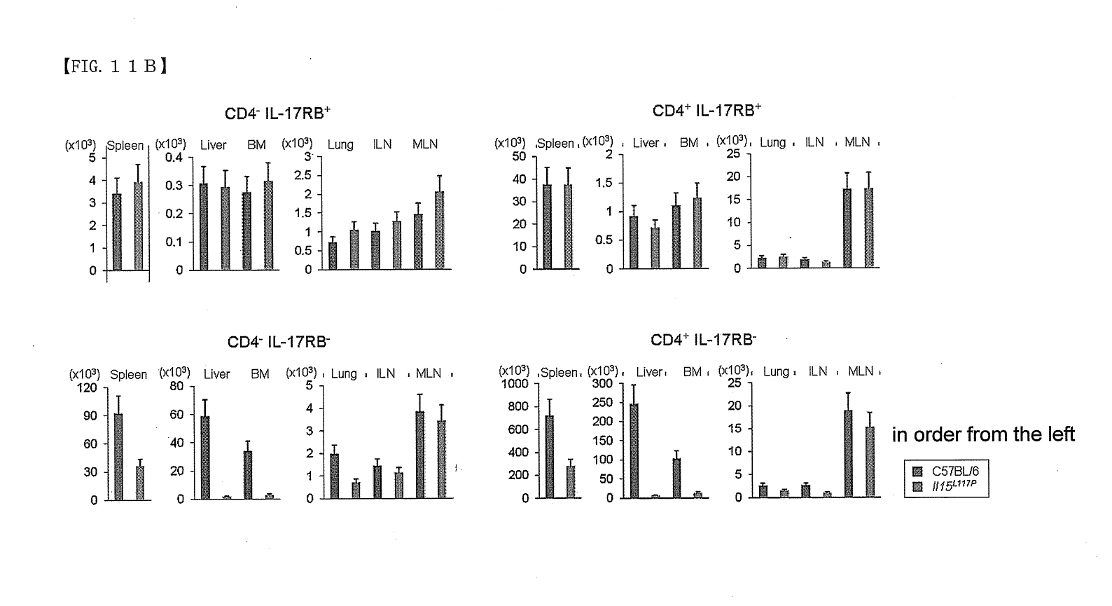

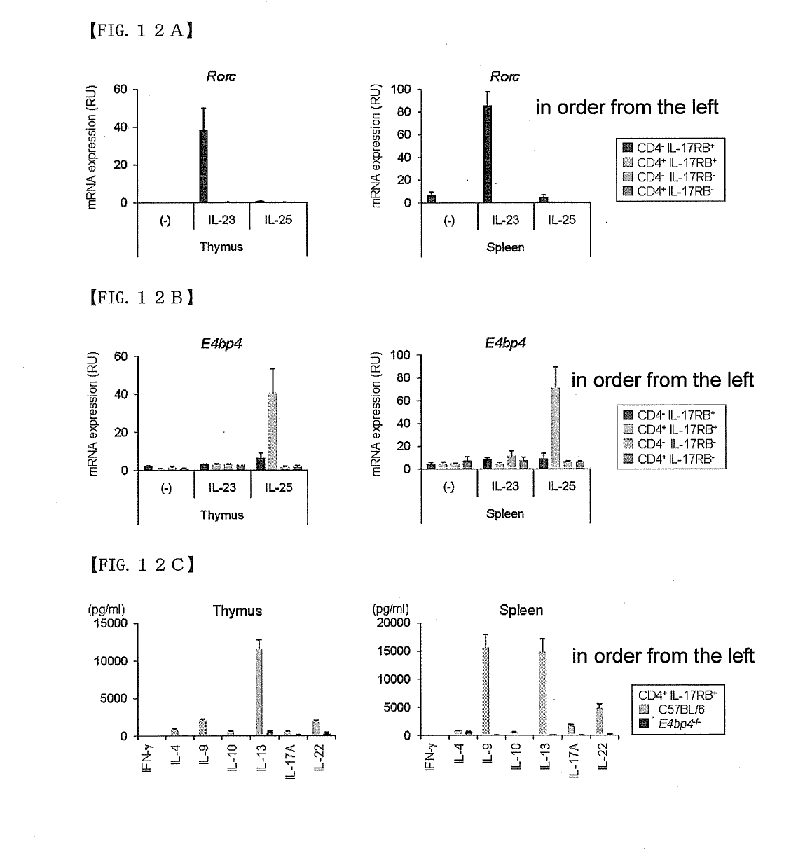

As for Th1/Th2/Th17 cytokine production by T cells and NKT cells, as transcription factors that control production thereof, T-bet, Gata3 and RORγt have been reported as master genes for Th1 cytokine IFN-γ, Th2 cytokine IL-4, and Th17 cytokine IL-17A, respectively. These transcription factors are known to be induced by stimulation with different cytokines, where IL-12 increases expression of T-bet, IL-4 increases expression of GATA3, and IL-23 increases expression of RORγt, to activate their functions. CD4 negative IL-17RB positive NKT cells, which are IL-17 producing NKT cells, express RORγt in a steady state and show increased expression by stimulation with IL-23, and therefore, IL-17A production by this subtype is considered to be RORγt dependent (FIG. 12A). On the other hand, while CD4 positive IL-17RB positive NKT cells produce Th2 cytokine and Th17 cytokine by stimulation with IL-25, the functional expression mode thereof is unknown. In fact, induction of RORγt does not occur in CD4 positive IL-17RB positive NKT cells even by stimulation with IL-25 (FIG. 12A), which suggests the presence of a mechanism that produces RORγt-independent Th17 cytokine.

In this Example, an increased expression of transcription factor E4 bp4 was found in CD4 positive IL-17RB positive NKT cells by the stimulation with IL-25 (FIG. 12B). Such increased expression is not observed in other subtypes by stimulation with IL-23 or IL-25 (FIG. 12B). Thus, cytokine production associated with stimulation, with IL-25, of CD4 positive IL-17RB positive NKT cells derived from the thymus and spleen of wild-type and E4 bp4 deficient mice was confirmed. As a result, a remarkable attenuation of the production of IL-4, IL-9, IL-10, IL-13, IL-17A, IL-22 was found in E4 bp4 deficient mice-derived CD4 positive IL-17RB positive NKT cells (FIG. 12C). Therefore, E4 bp4 is considered to play an important role in the induction of production of not only Th2 cytokine but also Th17 cytokine in CD4 positive IL-17RB positive NKT cells.

### Example 6

### Onset Mechanism of Hyperesponsive Airway Due to RS Virus Infection

RS virus is considered to have the key role in the onset of viral asthma particularly in childhood. However, since the onset mechanism thereof is complicated and diverse, detailed molecular mechanism, expression control and the like contain many unclear aspects. Thus, we tried to establish a mice model using soluble form of G protein (Gs) considered to be responsible for the onset of airway inflammation by RS virus.

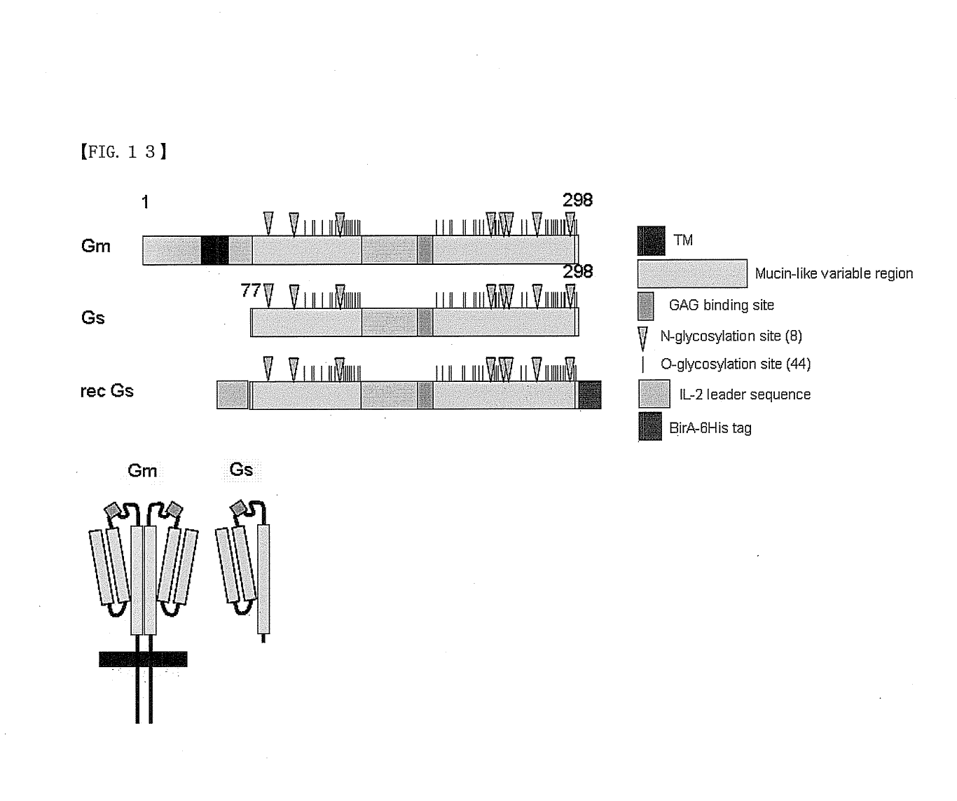

Membrane form of G protein (Gm) is a type II membrane protein consisting of 298 amino acids, and Gs consists of an extracellular domain (77-298) of Gm. Gs contains two mucin-like variable regions, and is a sugar chain rich protein having 8 N-linked sugar sites and 44 O linked sugar sites (FIG. 13). When a recombinant is expressed in an animal cell, 25-120 kDa glycoprotein is obtained.

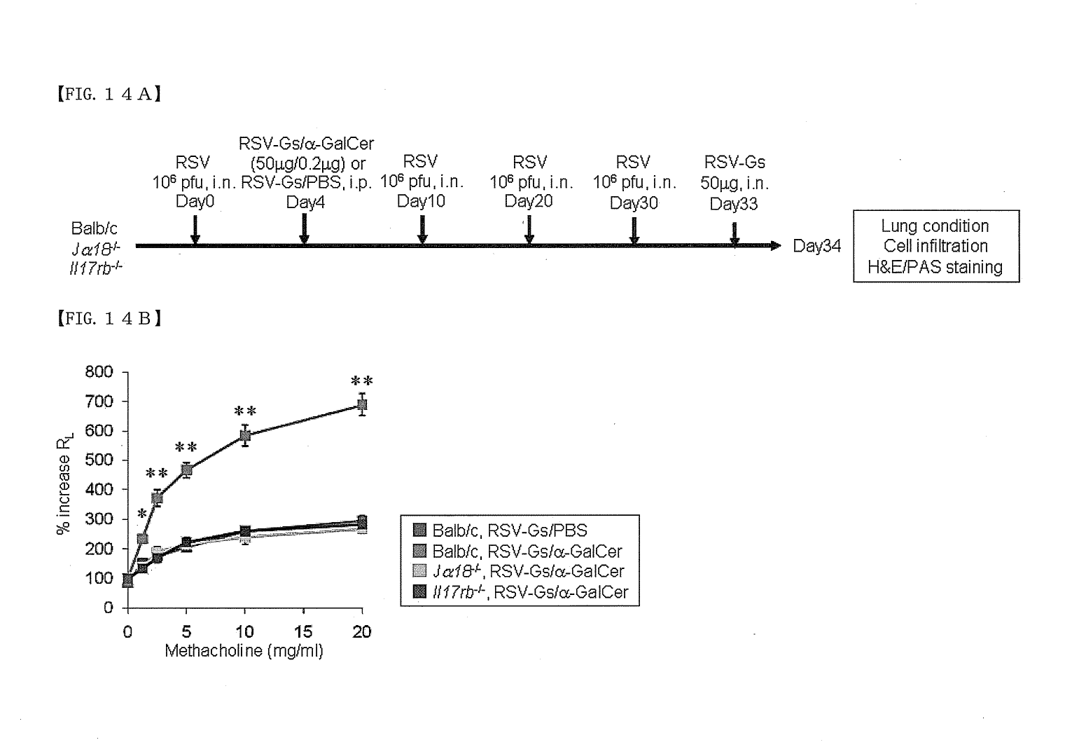

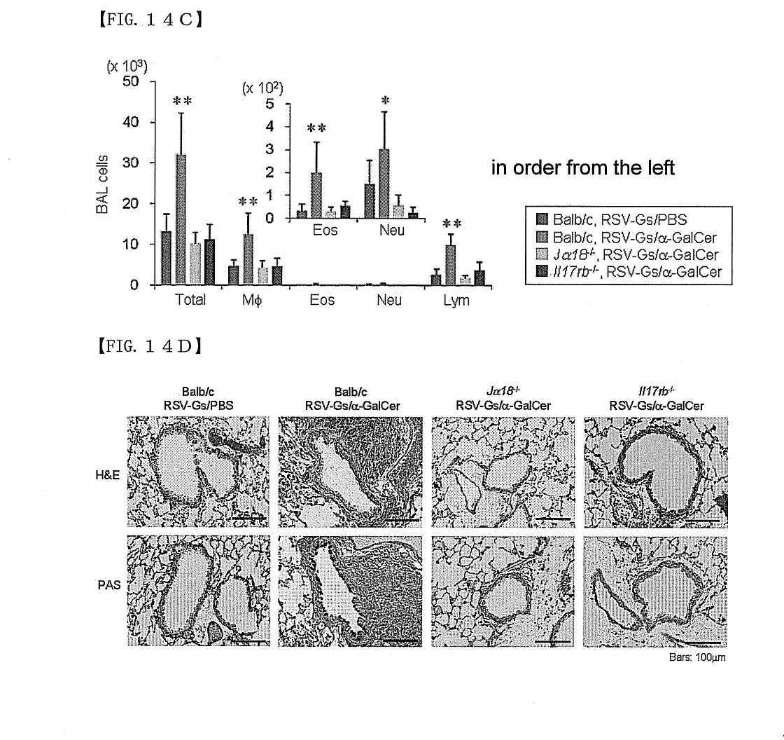

Wild-type and NKT cell deficient Jα18 deficient mice, and IL-17rb deficient mice (Balb/c background) were transnasally infected with 10⁶ pfu RS virus (A2 strain) 4 times at 10 day intervals. After 4 days from the initial infection, the mice were intraperitoneal immunized with Gs (50 μg) and administered with α-GalCer (0.2 μg) and transnasally administered with Gs 3 days after the final infection. The next day, the airway pressure and cell infiltration were observed (FIG. 14A). Increase of methacholine-induced airway pressure was significantly high in the group of wild-type mice immunized with Gs and administered with α-GalCer (FIG. 14B). In this model, activation of NKT cells is important, and the increase of airway pressure was markedly low when saline was used instead of α-GalCer or NKT cell deficient mice were used (FIG. 14B).

Since an airway pressure increase, rate is almost the same even in IL-17rb deficient mice, IL-17RB positive NKT cells are suggested to play a central role in the increase of airway pressure in this model. A detailed analysis of infiltrated cells in bronchoalveolar washing has confirmed infiltration of mainly macrophages and lymphocytes (FIG. 14C). In this model, infiltration of eosinophils and neutrophils is significant in wild-type mice immunized with Gs and administered with α-GalCer, but the number of cells is extremely small. Such results suggest possibility of induction by a mechanism different from eosinophils infiltration generally observed in allergy-induced airway hyperreactivity. In fact, from the results of H&E (hematoxylin-eosin) staining, and PAS staining (Periodic acid-Schiff reaction), excess production of mucin and cell infiltration were confirmed (FIG. 14D).

H&E staining is a tissue-specific staining method, where hematoxylin stains cell nucleus, bone tissue, partial cartilage tissue, serous fluid component and the like, and eosin stains cytoplasm, bond tissue of soft tissue, red blood cell, fibrin, endocrine granule and the like. PAS staining is a staining method that mainly stains glycogen, and cytoplasmic glycogen granules, secretion from apocrine gland and the like, foreign organism in the body such as bacterium, parasite and the like, keratohyaline granule and the like test positive to PAS staining, and collagen fiber, blood vessel endothelium and the like test weakly positive to PAS staining.

### INDUSTRIAL APPLICABILITY

Activated IL-17RB positive NKT cells play a central role in the aggravation of non-allergic airway hyperreactivity and non-allergic airway inflammation. Therefore, respiratory diseases are expected to be treated by inhibiting or removing the function of IL-17RB positive NKT cells.

This application is based on a patent application No. 61/553,544 filed in the US (filing date: Oct. 31, 2011), the contents of which are incorporated in full herein.

1. A method of preventing or treating non-allergic airway inflammation and/or non-allergic airway hyperreactivity, comprising administering an effective amount of a substance capable of inhibiting or removing Th17 cell-like function of IL-17RB positive NKT cells to a mammal.

2. The method according to claim 1, wherein the substance capable of inhibiting or removing Th17 cell-like function of IL-17RB positive NKT cells is at least one kind selected from the group consisting of an antagonistic antibody and a low-molecular inhibitor to IL-17RB, an antagonistic antibody and a low-molecular inhibitor to IL-23R, an antibody to IL-25 and a soluble molecule of IL-17RB, and an antibody to IL-23 and a soluble molecule of IL-17RB.

3. A method of screening for a therapeutic agent for non-allergic airway inflammation and/or non-allergic airway hyperreactivity, comprising the step of measuring the Th17 cell-like function of an IL-17RB positive NKT cells.

4. A method of screening for a therapeutic agent for non-allergic airway inflammation and/or non-allergic airway hyperreactivity, comprising the steps of:
contacting an IL-17RB positive NKT cells with a test compound in the presence of a ligand (step 1),
measuring the Th17 cell-like function of the IL-17RB positive NKT cells contacted with the test compound and the Th17 cell-like function of an IL-17RB positive NKT cells not being contacted with the test compound, and comparing the results (step 2), and
selecting a test compound significantly inhibiting the Th17 cell-like function as a therapeutic agent for non-allergic airway inflammation and/or non-allergic airway hyperreactivity (step 3).

5. The method according to claim 4, wherein the Th17 cell-like function is the ligand stimulation-dependent Th17 cytokine/chemokine producing ability.

6. The method according to claim 4, wherein the ligand is IL-23.

7. The method according to claim 4, wherein the ligand is IL-25.

8. The method according to claim 5, wherein the Th17 cytokine chemokine is at least one kind selected from the group consisting of IL-17A, IL-17F, IL-21, and IL-22.

9. A method of screening for a therapeutic agent for non-allergic airway inflammation and/or non-allergic airway hyperreactivity, comprising the steps of:
administering a test compound to a mammal other than human, having an IL-17RB positive NKT cells in the presence of a ligand of IL-17RB (step 1),
measuring the Th17 cell-like function of the mammal to which the test compound is administered, and the Th17 cell-like function of a mammal to which the test compound is not administered, and comparing the results (step 2), and
selecting a test compound significantly inhibiting the Th17 cell-like function as a therapeutic agent for non-allergic airway inflammation and/or non-allergic airway hyperreactivity (step 3).

10. The method according to claim 9, wherein the Th17 cell-like function is the ligand stimulation-dependent Th17 cytokines/chemokines producing ability.

11. The method according to claim 9, wherein the ligand is IL-23.

12. The method according to claim 9, wherein the ligand is IL-25.

13. The method according to claim 10, wherein the Th17 cytokine/chemokine is at least one kind selected from the group consisting of IL-17A, IL-17F, IL-21, and IL-22.

14. The method according to claim 5, wherein the ligand is IL-23.

15. The method according to claim 5, wherein the ligand is IL-25.

16. The method according to claim 10, wherein the ligand is IL-23.

17. The method according to claim 10, wherein the ligand is IL-25.


SEQUENCE LISTING (68 sequences)

SEQ ID NO: 1 - DNA, 23 bp, Artificial
  Forward primer (Cd4)
ctgactctgactctggacaaagg

SEQ ID NO: 2 - DNA, 21 bp, Artificial Sequence
  Reverse primer (Cd4)
ggagaggtaggtcccatcacc

SEQ ID NO: 3 - DNA, 28 bp, Artificial Sequence
  Probe (Cd4)
ttgagctgagccactttcatcaccacca

SEQ ID NO: 4 - DNA, 21 bp, Artificial Sequence
  Forward primer (Il17rb)
ccagatgacaacagacgcatg

SEQ ID NO: 5 - DNA, 23 bp, Artificial Sequence
  Reverse primer (Il17rb)
gagcatggtggaaataggaaagg

SEQ ID NO: 6 - DNA, 26 bp, Artificial Sequence
  Probe (Il17rb)
cgtcttcgtgctccttccttgcctcc

SEQ ID NO: 7 - DNA, 22 bp, Artificial Sequence
  Forward primer (Il2rb)
gaagggttggcgtagggtaaag

SEQ ID NO: 8 - DNA, 22 bp, Artificial Sequence
  Reverse primer (Il2rb)
gcagaacttggagggaatgagg

SEQ ID NO: 9 - DNA, 26 bp, Artificial Sequence
  Probe (Il2rb)
tccctttgacaaccttcgcctggtgg

SEQ ID NO: 10 - DNA, 25 bp, Artificial Sequence
  Forward primer (Ifng)
ggatgcattcatgagtattgccaag

SEQ ID NO: 11 - DNA, 21 bp, Artificial Sequence
  Reverse primer (Ifng)
ctccttttccgcttcctgagg

SEQ ID NO: 12 - DNA, 25 bp, Artificial Sequence
  Probe (Ifng)
aggtcaacaacccacaggtccagcg

SEQ ID NO: 13 - DNA, 23 bp, Artificial Sequence
  Forward primer (Tbx21)
aaggattccgggagaactttgag

SEQ ID NO: 14 - DNA, 24 bp, Artificial Sequence
  Reverse primer (Tbx21)
tggttggatagaagaggtgagaag

SEQ ID NO: 15 - DNA, 30 bp, Artificial Sequence
  Probe (Tbx21)
tgtacgcatctgttgatacgagtgtcccct

SEQ ID NO: 16 - DNA, 23 bp, Artificial Sequence
  Forward primer (Stat4)
agggaagagaggagaatattggc

SEQ ID NO: 17 - DNA, 24 bp, Artificial Sequence
  Reverse primer (Stat4)
gttccacattcctttgtctttcag

SEQ ID NO: 18 - DNA, 26 bp, Artificial Sequence
  Probe (Stat4)
cagccaacatgcctatccagggacct

SEQ ID NO: 19 - DNA, 22 bp, Artificial Sequence
  Forward primer (Il4)
catcggcattttgaacgaggtc

SEQ ID NO: 20 - DNA, 20 bp, Artificial Sequence
  Reverse primer (Il4)
cgttgctgtgaggacgtttg

SEQ ID NO: 21 - DNA, 25 bp, Artificial Sequence
  Probe (Il4)
tctccgtgcatggcgtcccttctcc

SEQ ID NO: 22 - DNA, 20 bp, Artificial Sequence
  Forward primer (Gata3)
gctacggtgcagaggtatcc

SEQ ID NO: 23 - DNA, 19 bp, Artificial Sequence
  Reverse primer (Gata3)
tccagccagggcagagatc

SEQ ID NO: 24 - DNA, 22 bp, Artificial Sequence
  Probe (Gata3)
cgacccaccacgggagccaggt

SEQ ID NO: 25 - DNA, 19 bp, Artificial Sequence
  Forward primer (Il17a)
ccttggcgcaaaagtgagc

SEQ ID NO: 26 - DNA, 24 bp, Artificial Sequence
  Reverse primer (Il17a)
atatctatcagggtcttcattgcg

SEQ ID NO: 27 - DNA, 26 bp, Artificial Sequence
  Probe (Il17a)
actacctcaaccgttccacgtcaccc

SEQ ID NO: 28 - DNA, 21 bp, Artificial Sequence
  Forward primer (Il22)
agcttgaggtgtccaacttcc

SEQ ID NO: 29 - DNA, 21 bp, Artificial Sequence
  Reverse primer (Il22)
aacagtttctccccgatgagc

SEQ ID NO: 30 - DNA, 24 bp, Artificial Sequence
  Probe (Il22)
agccgtacatcgtcaaccgcacct

SEQ ID NO: 31 - DNA, 22 bp, Artificial Sequence
  Forward primer (Rorc)
ggctttccatcatcatctctgc

SEQ ID NO: 32 - DNA, 20 bp, Artificial Sequence
  Reverse primer (Rorc)
ggtggaggtgctggaagatc

SEQ ID NO: 33 - DNA, 26 bp, Artificial Sequence
  Probe (Rorc)
cctcctagccaagctgccacccaaag

SEQ ID NO: 34 - DNA, 20 bp, Artificial Sequence
  Forward primer (Il17ra)
gtgccctgcccagtaatctc

SEQ ID NO: 35 - DNA, 21 bp, Artificial Sequence
  Reverse primer (Il17ra)
atggcgatgagtgtgatgagg

SEQ ID NO: 36 - DNA, 26 bp, Artificial Sequence
  Probe (Il17ra)
accacagttcccaagccagttgcaga

SEQ ID NO: 37 - DNA, 18 bp, Artificial Sequence
  Forward primer (Il12rb1)
cgctgcgaggctgaagac

SEQ ID NO: 38 - DNA, 20 bp, Artificial Sequence
  Reverse primer (Il12rb1)
cgcagtccgtcaagtgtcac

SEQ ID NO: 39 - DNA, 24 bp, Artificial Sequence
  Probe (Il12rb1)
cacgagccactctgactcccacgc

SEQ ID NO: 40 - DNA, 19 bp, Artificial Sequence
  Forward primer (Il12rb2)
cgcttctgcacccactcac

SEQ ID NO: 41 - DNA, 23 bp, Artificial Sequence
  Reverse primer (Il12rb2)
tgccaggtcactagaatgttgtc

SEQ ID NO: 42 - DNA, 24 bp, Artificial Sequence
  Probe (Il12rb2)
cactgggttgctggctcctcacca

SEQ ID NO: 43 - DNA, 25 bp, Artificial Sequence
  Forward primer (Il23r)
gcttctactacatttgggacatgag

SEQ ID NO: 44 - DNA, 20 bp, Artificial Sequence
  Reverse primer (Il23r)
caccaggctcaacccacatg

SEQ ID NO: 45 - DNA, 30 bp, Artificial Sequence
  Probe (Il23r)
tgattcctccgtgacaccatctgaagagca

SEQ ID NO: 46 - DNA, 23 bp, Artificial Sequence
  Foward primer (Ccr4)
ctcaggatcactttcagaagagc

SEQ ID NO: 47 - DNA, 20 bp, Artificial Sequence
  Reverse primer (Ccr4)
ggtggtgtctgtgacctctg

SEQ ID NO: 48 - DNA, 26 bp, Artificial Sequence
  Probe (Ccr4)
aggcagctcaactgttctcattggct

SEQ ID NO: 49 - DNA, 22 bp, Artificial Sequence
  Forward primer (Ccr6)
ctgcccacttccctttctacac

SEQ ID NO: 50 - DNA, 25 bp, Artificial Sequence
  Reverse primer (Ccr6)
ctgtgttgtcataatcatccgttcc

SEQ ID NO: 51 - DNA, 25 bp, Artificial Sequence
  Probe (Ccr6)
tcattccccaggcaggcgtggttct

SEQ ID NO: 52 - DNA, 19 bp, Artificial Sequence
  Forward primer (Ccr7)
catggacccagggaaaccc

SEQ ID NO: 53 - DNA, 21 bp, Artificial Sequence
  Reverse primer (Ccr7)
tgacctcatcttggcagaagc

SEQ ID NO: 54 - DNA, 26 bp, Artificial Sequence
  Probe (Ccr7)
tgacaaggagagccaccaccagcacg

SEQ ID NO: 55 - DNA, 19 bp, Artificial Sequence
  Forward primer (Cxcr3)
gaagcaggcagcacgagac

SEQ ID NO: 56 - DNA, 21 bp, Artificial Sequence
  Reverse primer (Cxcr3)
ccgaggcatctagcacttgac

SEQ ID NO: 57 - DNA, 25 bp, Artificial Sequence
  Probe (Cxcr3)
cggagcaccagccaagccatgtacc

SEQ ID NO: 58 - DNA, 23 bp, Artificial Sequence
  Forward primer (Cxcr6)
cacacttcactctggaacaaagc

SEQ ID NO: 59 - DNA, 24 bp, Artificial Sequence
  Reverse primer (Cxcr6)
tggctgttatcactggaattgttg

SEQ ID NO: 60 - DNA, 29 bp, Artificial Sequence
  Probe (Cxcr6)
agccagaaatctccctcgtagtgcccatc

SEQ ID NO: 61 - DNA, 20 bp, Artificial Sequence
  Forward primer (E4bp4)
acagccgccctttcttttcc

SEQ ID NO: 62 - DNA, 22 bp, Artificial Sequence
  Reverse primer (E4bp4)
ggacttcagcctctcatccatc

SEQ ID NO: 63 - DNA, 26 bp, Artificial Sequence
  Probe (E4bp4)
accagggagcagaaccacgataaccc

SEQ ID NO: 64 - DNA, 28 bp, Artificial Sequence
  Forward primer (Hprt1)
gaggatttggaaaaagtgtttattcctc

SEQ ID NO: 65 - DNA, 20 bp, Artificial Sequence
  Reverse primer (Hprt1)
gatggcctcccatctccttc

SEQ ID NO: 66 - DNA, 30 bp, Artificial Sequence
  Probe (Hprt 1)
catctcgagcaagtctttcagtcctgtcca

SEQ ID NO: 67 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 68 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK